(19) United States
(12) Patent Application Publication
(10) Pub. No.: US 2025/0042887 A1
(43) Pub. Date: Feb. 6, 2025

(54) GLYCOGEN SYNTHASE KINASE-3 (GSK3) INHIBITOR COMPOUNDS FOR USE AS ANTIVIRAL AGENTS

(71) Applicant: THE UNIVERSITY OF BRITISH COLUMBIA, Vancouver (CA)

(72) Inventors: Yossef AV-GAY, Vancouver (CA); Tirosh SHAPIRA, Vancouver (CA); Steven PELECH, Richmond (CA); François JEAN, Vancouver (CA)

(21) Appl. No.: 18/716,093

(22) PCT Filed: Dec. 7, 2022
(86) PCT No.: PCT/CA2022/051784

§ 371 Date: Jun. 3, 2024

Related U.S. Application Data
(60) Provisional application No. 63/286,632, filed on Dec. 7, 2021.

(51) Int. Cl. C07D 413/04; A61K 31/4245; A61P 31/14

(52) U.S. Cl. CPC C07D 413/04; A61K 31/4245; A61P 31/14

(57) ABSTRACT

Provided herein are compounds that inhibit glycogen synthase kinase-3 (GSK3) giving them antiviral activity. In particular, the invention relates to a subset of compounds represented by Formulas (I) and (II), for use as antiviral agents in the treatment or prevention of viral infection. Methods for using the GSK3 inhibitor compounds in the treatment or prophylaxis of a viral infection are provided. In particular, the viral infection may be selected from one or more of the following: Severe Acute Respiratory Syndrome (SARS) coronavirus-1 SARS-CoV-1) infection; SARS coronavirus-2 (SARS-CoV-2) infection; and Middle East Respiratory Syndrome (MFRS) coronavirus (MERS-CoV) infection. More specifically, the viral infection may be a human coronavirus 229E (HCoV-229E) infection.

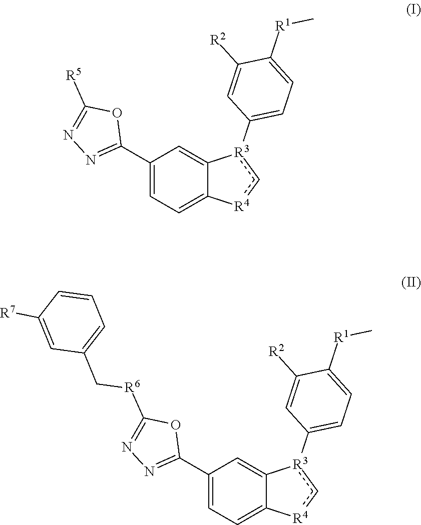

### CROSS REFERENCE TO RELATED APPLICATIONS

This application claims the benefit of U.S. Provisional Patent Application Ser. No. 63/286,632 filed 7 Dec. 2021 entitled “GSK3 COMPOUNDS AND ANTIVIRAL ACTIVITY”.

### TECHNICAL FIELD

The present invention relates to glycogen synthase kinase-3 (GSK3) inhibitor compounds having antiviral activity. In particular, the invention relates to a subset of compounds represented by Formulas I and II, for use as antiviral agents in the treatment or prevention of viral infection. Methods for using the GSK3 inhibitor compounds in the treatment or prophylaxis of a viral infection are provided. In particular, the viral infection may selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. More specifically, the viral infection may be a human coronavirus 229E (HCoV-229E) infection.

### BACKGROUND

Coronavirus disease 2019 (COVID-19) is caused by the novel coronavirus SARS-CoV-2, which has infected over 570 million people worldwide, been linked to over 6.4 million deaths, and precipitated significant global disruptions to social and economic structures [1, 2]. Due to our limited knowledge of the molecular details of SARS-CoV-2 pathogenesis, the majority of current antiviral therapeutic efforts are focused on either repurposing existing antiviral drugs or finding new drugs that target viral entry or replication.

Most infected patients clear SARS-CoV-2 infection without treatment, through mounting innate and adaptive immune responses, demonstrating that human hosts have a built-in capacity to neutralize and overcome acute infections [3]. At the molecular level, surveillance and response to infection is mediated by various cellular signaling pathways that activate protective host processes including autophagy, apoptosis or even necrosis of infected cells. Consequently, this research examined the modulation of host signaling proteins that enhance the cell's innate ability to clear infection and may be important for viral replication.

Signaling mediated responses to infection rely on protein-protein interactions (PPIs) and associated post-translational phosphorylation. A recent PPI study [4] analysing interactions between SARS-CoV-2 proteins and the human proteome identified over 300 high-confidence PPIs. Of the 300+ human proteins identified, many were protein signaling targets of approved, pre-clinical, or investigational drugs [4]. These PPIs, therefore, are potential candidates for use in host-directed therapies (HDTs), which alternatively target host pathways and processes rather than the virus. As viral infections hijack host functions, HDTs may interfere with these interactions or contribute to the host cell's ability to fight the infection. Additionally, as HDTs do not target the virus directly, there is an enhanced potential for these therapies to provide broader protection against various viral strains and alleviate the pressures leading to downstream drug resistance through viral mutations and selection.

Library screening of FDA-approved drugs for inhibitors of coronavirus replication identified Abelson (Abl) kinase inhibitors, including imatinib, as inhibitors of both the 2003 SARS-CoV strain and MERS-CoV in vitro [5]. Moreover, inhibition of another key signaling protein, Glycogen synthase kinase-3 (GSK3), was found to reduce viral nucleocapsid (N protein) phosphorylation in SARS-CoV-infected VeroE6 cells and decrease viral titer and cytopathic effects. The effect of the GSK3 inhibitor was reproduced in another coronavirus, the neurotropic strain of mouse hepatitis virus. These results indicate that GSK3 is critical for coronavirus N protein phosphorylation and indicate that GSK3 plays a role in regulating the viral life cycle [6]. Furthermore, SARS-CoV-2 N protein phosphorylation was recently shown to be abolished in GSK3α and GSK3β, knock down cells [7]. As such, GSK3β has been suggested as suitable candidate target for COVID-19 host-directed antiviral therapy [8].

The SARS-CoV-2 N protein is an abundant RNA-binding protein critical for viral genome packaging, yet the molecular processes and characteristics of this function are not fully understood. It acts as a phosphoprotein that associates with the viral RNA genome to form the ribonucleoprotein core [9]. Composed of three dynamic, disordered regions that house putative transiently-helical binding motifs, it adopts a conformation of two folded domains that interact minimally such that it remains a flexible and multivalent RNA-binding protein.

GSK3 is a key control kinase of glycogen synthesis [10] and is represented with the GSK3α and GSK3β isoforms [11], each with differential regulation [12] and tissue expression [13]. GSK3 serves a central signaling role [14] in many regulatory processes through intersection with the PI3K, mTOR, PKB/AKT, WNT, and MAPK pathways [15]. As such, GSK3 is currently a target for drug discovery efforts in Alzheimer's disease, cancer, diabetes, multiple sclerosis, and others (reviewed in [16]). GSK3β has additionally been shown to play a role in bacterial [17] and viral infection control [7; 47].

A recent study investigated the effect of kinase inhibitors against COVID-19, showed that Enzastaurin, a clinically well-tolerated protein-serine/threonine kinase inhibitor, reduced infection in the A549-ACE2 human lung cancer-derived cell line and Vero E6 cells[7]. The inhibitor at 10 mM blocked N protein phosphorylation and SARS-CoV-2 replication in cells, whereas only one tenth the concentration at 1 mM was required for GSK3 inhibition in humans [7]. Lithium chloride, a non-specific GSK3 inhibitor used to treat patients with bipolar disorder (BD), has also been shown to reduce the risk of developing COVID-19 compared to non-users [7], but current clinical therapeutic ranges have not been observed to be effective in vitro. An IC₅₀ of 10 mM was needed to inhibit N protein phosphorylation and SARS-CoV-2 replication in cells, whereas only a fraction, at 1 mM, is required for GSK3 inhibition in humans [7]. Thus, although GSK3 has been confirmed to play a role in N phosphorylation for viral replication, given GSK3 inhibition varied across conditions, further screening is required to identify better compounds that are effective against this host derived therapeutic target.

### SUMMARY

The present invention is based, in part, on the surprising discovery that a subset of compounds represented by Formulas I and II described herein, for use as antiviral agents in the treatment or prevention of viral infection. Methods for using the GSK3 inhibitor compounds in the treatment or prophylaxis of a viral infection are provided. In particular, the viral infection may selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. More specifically, the viral infection may be a human coronavirus 229E (HCoV-229E) infection.

In a first embodiment, there is provided a compound, the compound may have the structure of Formula I:

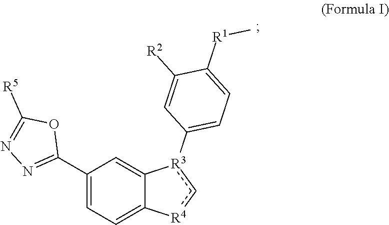

or pharmaceutical acceptable salt or prodrug thereof, for treatment of a viral infection, wherein:
[symbol] may be optionally a single or a double bond, provided that both are not double bonds and provided that both are not single bonds;
R¹ may be selected from: S; S═O; C; and SO₂;
R² may be selected from: H; halogen; OCF₃; CF₃; C₁₋₁₀-alkyl; and CN;
R³ may be selected from: C; and N;
R⁴ may be selected from: O; and N;
R₅ may be selected from: NH₂;

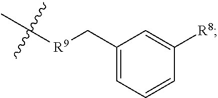

### and CH₃;

R⁸ may be selected from: CN; halogen; H; CF₃; C₁₋₁₀-alkyl; and OCF₃; and
R⁹ may be selected from: CH₂; S; and O.

Alternatively, the compound may have the structure of Formula II:

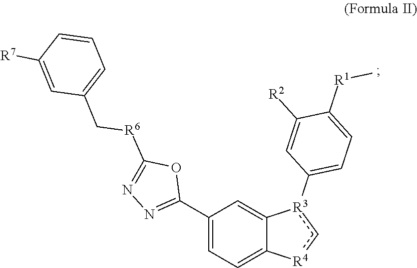

or pharmaceutically acceptable salt or prodrug thereof, for treatment of a viral infection, wherein:
[symbol] may be optionally a single or a double bond, provided that both are not double bonds and provided that both are not single bonds;
R¹ may be selected from: S; S═O; C; and SO₂;
R² may be selected from: H; halogen; OCF₃; CF₃; C₁₋₁₀-alkyl; and CN;
R³ may be selected from: C; and N;
R⁴ may be selected from: O; and N;
R⁶ may be selected from: CH₂; S; and O; and
R⁷ may be selected from: CN; halogen; H; CF₃; C₁₋₁₀-alkyl; and OCF₃.

Alternatively, the compound may have the structure:

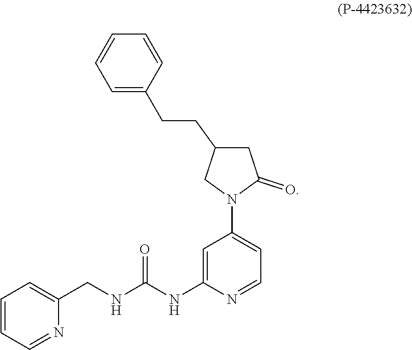 [SMILES: O=C(NCc1ccccn1)Nc1cc(N2CC(CCc3ccccc3)CC2=O)ccn1]

In a further embodiment, there is provided a method of treating a viral infection in a subject in need thereof, the method comprising administration of a compound to the subject, wherein the compound has the structure of Formula I or Formula II or P-4423632, as described herein.

In a further embodiment, there is provided a pharmaceutical composition, the pharmaceutical composition comprising a compound having the structure of Formula I or Formula II or P-4423632, as described herein.

In a further embodiment, there is provided a use of a compound; or a pharmaceutical composition comprising the compound and a pharmaceutically acceptable carrier; for treating a viral infection, the compound having the structure of Formula I or Formula II or P-4423632, as described herein.

In a further embodiment, there is provided a use of a compound in the manufacture of a medicament for treating a viral infection, the compound having the structure of Formula I or Formula II or P-4423632, as described herein.

The halogen may be selected from F, Cl, Br, and I. The halogen may be selected from F and Cl.

The halogen may be selected from F, Cl, and Br. The halogen may be F. The halogen may be Cl.

The halogen may be Br. The halogen may be I. The halogen may be selected from F, and Br.

The halogen may be selected from Cl, and Br.

R¹ may be selected from: S; and S═O. R¹ may be S. R¹ may be S═O.

R² may be selected from: H; F; Cl; and OCF₃. R² may be selected from: H; Cl; and OCF₃. R² may be selected from: H; F; and Cl. R² may be selected from: F; Cl; and OCF₃. R² may be selected from: H; and OCF₃. R² may be H; F; Cl; and OCF₃. R² may be H. R² may be F. R² may be Cl. R² may be OCF₃.

R³ may be selected from: C; and N. R³ may be N. R³ may be C.

R⁴ may be selected from: O; and N. R⁴ may be O. R⁴ may be N.

R⁵ may be selected from: NH₂;

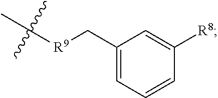

and CH₃. R⁵ may be selected from: NH₂; and

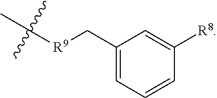

R⁵ may be selected from:

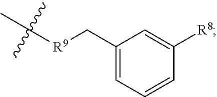

and CH₃. R⁵ may be selected from: NH₂; and CH₃. R⁵ may be

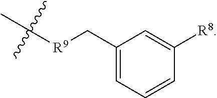

R⁵ may be NH₂. R⁵ may be CH₃.

R⁶ may be selected from: O; CH₂; and S. R⁶ may be selected from: CH₂; and S. R⁶ may be CH₂. R⁶ may be S. R⁶ may be O.

R⁷ may be selected from: CN; halogen; H; CF₃; C₁₋₁₀-alkyl; and OCF₃. R⁷ may be selected from: CN; F; Cl; Br; H; and CF₃. R⁷ may be selected from: CN; F; Cl; H; and CF₃. R⁷ may be selected from: CN; F; Cl; and CF₃. R⁷ may be selected from: CN; F; and CF₃. R⁷ may be selected from: CN; Cl; and CF₃. R⁷ may be selected from: CN; H; and CF₃. R⁷ may be selected from: CN; and CF₃. R⁷ may be selected from: CN; F; and Cl. R⁷ may be selected from: CN; and CF₃. R⁷ may be selected from: CN; Br; and CF₃. R⁷ may be selected from: F; Cl; Br; and CF₃. R⁷ may be selected from: F; Cl; and Br. R⁷ may be selected from: CN; F; Cl; Br; and CF₃. R⁸ may be CN. R⁷ may be F. R⁸ may be Cl. R⁷ may be Br. R⁷ may be CF₃. R⁷ may be H. R⁷ may be OCF₃.

R⁸ may be selected from: CN; halogen; H; CF₃; C₁₋₁₀-alkyl; and OCF₃. R⁸ may be selected from: CN; F; Cl; Br; H; and CF₃. R⁸ may be selected from: CN; F; Cl; H; and CF₃. R⁸ may be selected from: CN; F; Cl; and CF₃. R⁸ may be selected from: CN; F; and CF₃. R⁸ may be selected from: CN; Cl; and CF₃. R⁸ may be selected from: CN; H; and CF₃. R⁸ may be selected from: CN; and CF₃. R⁸ may be selected from: CN; F; and Cl. R⁸ may be selected from: CN; and CF₃. R⁸ may be selected from: CN; Br; and CF₃. R⁸ may be selected from: F; Cl; Br; and CF₃. R⁸ may be selected from: F; Cl; and Br. R⁸ may be selected from: CN; F; Cl; Br; and CF₃. R⁸ may be CN. R⁸ may be F. R⁸ may be Cl. R⁸ may be Br. R⁸ may be CF₃. R⁸ may be H. R⁸ may be OCF₃.

R⁹ may be selected from: O; CH₂; and S. R⁹ may be selected from: CH₂; and S. R⁹ may be CH₂. R⁹ may be S. R⁹ may be O.

R¹ may be selected from: S; and S═O; R² may be selected from: H; Cl; and OCF₃; R³ may be selected from: C; and N; R⁴ may be selected from: O; and N; R⁵ may be selected from: NH₂.

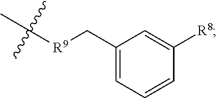

and CH₃; R⁸ may be selected from: CN; F; and CF₃; and R⁹ may be selected from: CH₂; and S.

The compound may be selected from one or more of:

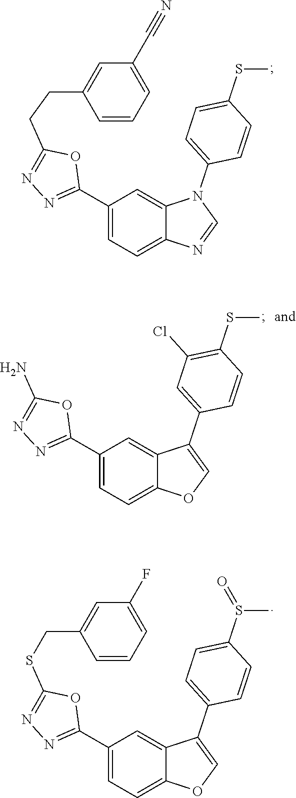

The compound may be

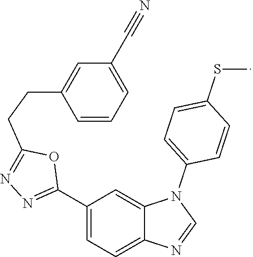

The compound may be

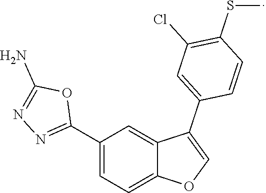

The compound may be

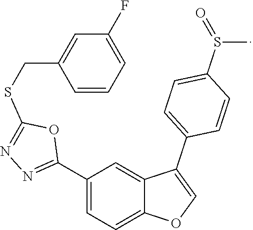

The compound may be selected from one or more of:

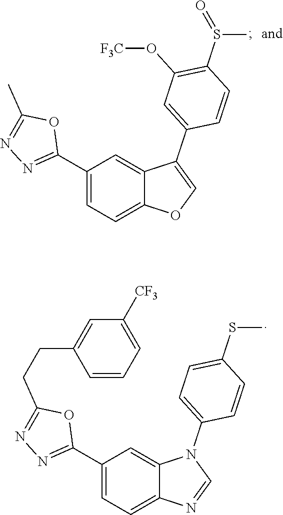

The compound may be

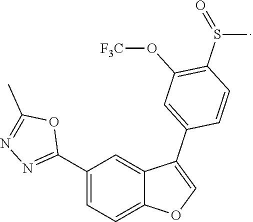

The compound may be

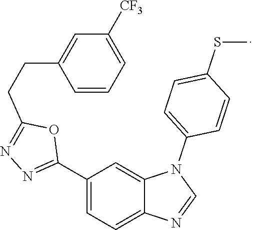

The compound may be selected from one or more of:

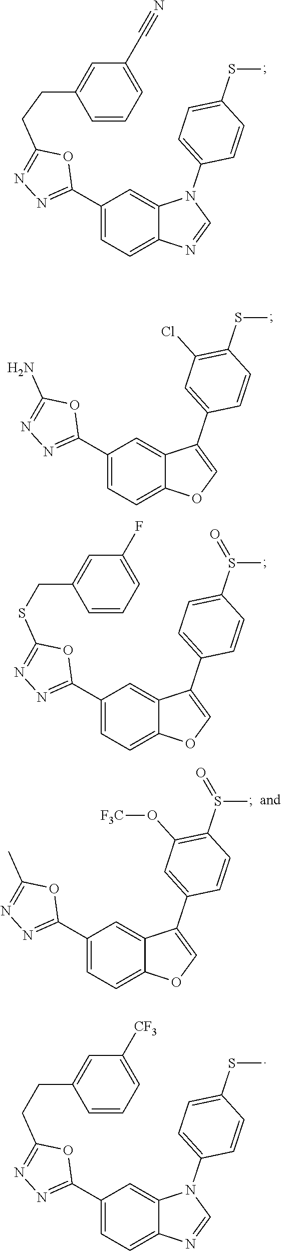

The viral infection may be selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. The viral infection may be human coronavirus 229E (HCoV-229E) infection. The viral infection may be a coronavirus infection. The coronavirus may be an alpha, a beta, a gamma, or a delta coronavirus. The coronavirus may be an α-coronavirus or a β-coronavirus. The viral infection may be selected from: a SARS-CoV-1 infection; a SARS-CoV-2 infection; and a MERS-CoV infection. The viral infection may be selected from: a SARS-CoV-1 infection; and a SARS-CoV-2 infection. The viral infection may be selected from: a SARS-CoV-1 infection; and a MERS-CoV infection. The viral infection may be selected from: a SARS-CoV-2 infection; and a MERS-CoV infection. The viral infection may be a SARS-CoV-1 infection. The viral infection may be a SARS-CoV-2 infection. The viral infection may be a MERS-CoV infection.

The treating of the viral infection might benefit from the inhibition of glycogen synthase kinase 3 (GSK3). The GSK3 may be GSK3β. The treating of the viral infection might benefit from the inhibition of N protein phosphorylation in a virus. The treating of the viral infection may include a virus that comprises a nucleocapsid N protein. The compound may inhibit N protein phosphorylation in the virus.

### BRIEF DESCRIPTION OF THE DRAWINGS

FIG. 1. shows (a) High-content screen of SARS-CoV-2 (full circles) and HCoV-229E (empty circles) infected Huh-7.5.1 cells with the Takeda GSK3β-focused library at a single dose of 10 μM. Inhibition was interpolated to both a non-infected control and an infected, untreated control, quantifying intracellular dsRNA. Cell loss was assessed to avoid quantifying small populations and compounds resulting in >25% cell loss are marked in red. Compounds marked with an asterisk were further pursued. The Z′ of two independent screens were 0.54 and 0.74 for SARS-CoV-2 and 0.68 for HCoV-229E. (b-c) Dose-response validation of compounds of interest. Each dose is an average of four independent experiments, error bars show SEM. Viral load of SARS-CoV-2 (b) was determined by measuring intracellular N protein and viral load of HCoV-229E (c) by measuring dsRNA. (d) Western analysis of SARS-CoV-2 N protein, representative blot of four independent experiments. (e) Representative synergy isoblogram of P-0717632 and Remdesivir analyzed with HCoV-229E. (f) Relationship between inhibition readout using dsRNA and SARS-CoV-2 nucleocapsid for all screened compounds. S=simple linear regression analysis resulted in a y=1.067× relationship, with 95% CI of 0.9741 to 1.160. (g) shows GSK3β inhibitor activity with (grey) and without (white) pre-treatment of five (5) active inhibitors (i.e. P-0717632 (T-1686568); P-3817632; P-5782442; P-7657632; and P-9071942) as compared to 0.1% DMSO and mock infection controls. Inhibition measured using a H—CoV-229E model of infection, capturing dsRNA immunofluorescence two days post infection. Inhibition interpolated to mock infection and media control. Error bars=SD from three experiments.

FIG. 2 shows the presence of SARS-CoV-2 proteins in infected Huh-7.5.1 cells treated with and without P-0717632. (a, c, e) Cells were pre-treated with 10 μM P-0717632 for 3 hours and then incubated with the virus for 2 days prior to harvesting and western blotting. (b, d, f) Recombinant preparations of SARS-CoV-2 protein and peptides were robotically spotted on to nitrocellulose membranes as configured in (g) and then immunoblotted with the following antibodies that were generated against peptides patterned after sequences in: (a, b) the nucleocapsid (Nucl.; NNCOV2N-1 raised against amino acids 156-170); (c, d) the non-structural protein NSP2 in the ORF1; NNCOV2-1A-2 raised against amino acids 735-750); and the spike protein S1 subunit (Spk S1; NNCOV2S-10 raised against amino acids 574-588). Locations of expected positions of target proteins and peptides are circled in the dot blots shown. Examples shown are representative of triplicate experiments. (h) Confocal images of Huh-7.5.1 infected with SARS-CoV-2 with or without 10 μM P-0717632 treatment for 48 h. Host nuclei (DAPI column and faint background in merge column), nucleocapsid (second column and merge column), spike (third column and merge column) are shown, with scale bar (white)=20 m. (i) Quantification of fluorescent nucleocapsid and spike proteins is averaged from three independent experiments, Student t-test * p<0.05.

FIG. 3 shows (a) dose-response inhibition effect of P-0717632 (T-1686568) in three infected immortalized cell lines, with data from four independent experiments; error bars are the SEM. (b) Viral titer from 48-hour infected Calu-3 cells, with (100 μM) and without (DMSO) P-0717632 (T-1686568) from 12 independent infections; error bars are the SEM. One-way ANOVA with Bonferroni multiple comparison correction, P<0.0001. LOD—Limit of Detection. (c) Semi-dose-response inhibition effect of P-0717632 (T-1686568) in primary donor-derived colon organoids, 72-hours post infection. Viral load interpolated to both anon-infected cells (mock) control and an infected, untreated control, quantifying intracellular N protein.

FIG. 4 shows confirmed phosphorylation sites in SARS-CoV-2 nucleocapsid protein (i.e. represented herein by MSDNGP (SEQ ID NO:9); APRITFGGP (SEQ ID NO:10); GPTDSTGSNQNGERSGARS (SEQ ID NO:11); ASWFTALTQ (SEQ ID NO:12); VPINTNSSPDDQ (SEQ ID NO:13); YRRATRRIR (SEQ ID NO:14); MKDLSPRWYFYYLGTGPEA (SEQ ID NO:15); GALNTPKDH (SEQ ID NO:16); PQGTTLPKG (SEQ ID NO:17); YAEGSRGGSQASSRSSSRSRNSSRNSTPGSSRGTSPARM (SEQ ID NO:18); and ESKMSGKGQVTKKSAAEAGMEVTPAGAKKQQTVTLLQQSMSSADSTQA (SEQ ID NO:19)), where the phosphosites referenced from before 2020 correspond to sites that were originally identified in the SARS-CoV-1 nucleocapsid protein, but are numbered according to their position in the SARS-CoV-2 nucleocapsid protein. These phosphosites were originally identified by mass spectrometry.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

GSK3β has been proposed to have an essential role in Coronaviridae infections. Screening of a targeted library of GSK3β inhibitors against both SARS-CoV-2 and HCoV-229E resulted in the identification of a number of active compounds, some with low toxicity to host cells. A particular compound of interest is P-0717632 (T-1686568), showed low micromolar, dose-dependent activity against SARS-CoV-2 and HCoV-229E. P-0717632 showed efficacy in cultured cells and primary organoids, reduction of the host cell's syncytia formation and filopodial protrusions. P-0717632 also inhibited SARS-CoV-2 variants of concern Delta and Omicron. Importantly, while inhibition by P-0717632 resulted in the overall reduction of viral load and protein translation, GSK3β inhibition resulted in cellular accumulation of the nucleocapsid protein. Protein kinase substrate profiling assays combined with Western blotting analysis showed that the SARS-CoV-2 nucleocapsid was phosphorylated by GSK3β on the S180/S184, S190/S194 and T198 phospho-sites, following previous priming in the adjacent S188, T198 and S206, respectively. Such inhibition presents a compelling target for broad-spectrum anti-Coronaviridae compound development, and underlies the mechanism of action of GSK3β host-directed therapy against this class of obligate intracellular pathogens.

Furthermore, compounds P-4423632, P-9071942, P-5908342, P-5782442, P-7657632, and P-3817632), were also found to have GSK3β inhibitory activity.

Any terms not directly defined herein shall be understood to have the meanings commonly associated with them as understood within the art of the invention.

It will be understood by a person of skill that NR₂ may include the corresponding ion, for example, ammonium ions. Alternatively, where the ions are shown, a person of skill in the art will appreciate that the counter ion may also be present.

Those skilled in the art will appreciate that the point of covalent attachment of the moiety to the compounds as described herein may be, for example, and without limitation, cleaved under specified conditions. Specified conditions may include, for example, and without limitation, in vivo enzymatic or non-enzymatic means. Cleavage of the moiety may occur, for example, and without limitation, spontaneously, or it may be catalyzed, induced by another agent, or a change in a physical parameter or environmental parameter, for example, an enzyme, light, acid, temperature or pH. The moiety may be, for example, and without limitation, a protecting group that acts to mask a functional group, a group that acts as a substrate for one or more active or passive transport mechanisms, or a group that acts to impart or enhance a property of the compound, for example, solubility, bioavailability or localization.

In some embodiments, compounds as described herein, may be used for the treatment of viral infection. Alternatively, the compounds described herein may be used to interfere with viral RNA processing including splicing, polyadenylation, and export to the cytoplasm. Alternatively, the compounds that may be suitable for the treatment of Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection. The viral infection may be the viral infection is human coronavirus 229E (HCoV-229E) infection.

Compounds as described herein may be in the free form or in the form of a salt thereof. In some embodiment, compounds as described herein may be in the form of a pharmaceutically acceptable salt, which are known in the art [50]. Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, i-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

In some embodiments, compounds and all different forms thereof (e.g. free forms, salts, polymorphs, hydrates, hydrated salts, optical isomers, racemates, diastereoisomers, enantiomers, isomeric forms) as described herein may be in a solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formulas illustrated for the sake of convenience.

In some embodiments, pharmaceutical compositions as described herein may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents (used interchangeably herein) are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein micro-particles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20th ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene polyoxypropylene copolymers may be used to control the release of the compounds. Other potentially useful parenteral delivery systems for modulatory compounds include ethylene vinyl acetate copolymer particles, osmotic pumps, implantable infusion systems, and liposomes. Formulations for inhalation may contain excipients, for example, lactose, or may be aqueous solutions containing, for example, polyoxyethylene 9 lauryl ether, glycocholate and deoxycholate, or may be oily solutions for administration in the form of nasal drops, or as a gel.

Compounds or pharmaceutical compositions as described herein or for use as described herein may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition as described herein includes a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors, increased life span, increased life expectancy or prevention of the progression of prostate cancer to an androgen independent form. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds as described herein should be used without causing substantial toxicity. Toxicity of the compounds as described herein can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be appropriate to administer substantial excesses of the compositions. Some compounds as described herein may be toxic at some concentrations. Titration studies may be used to determine toxic and non-toxic concentrations. Animal studies may be used to provide an indication if the compound has any effects on other tissues.

Compounds as described herein may be administered to a subject. As used herein, a “subject” may be a human, non-human primate, rat, mouse, cow, horse, pig, sheep, goat, dog, cat, etc. The subject may be suspected of having or at risk for having a viral infection, such as SARS-CoV-1, SARS-CoV-2, and MERS-CoV. Alternatively, the infection may be a coronavirus infection. Diagnostic methods for various viral infections, are known to those of ordinary skill in the art.

As used herein, term “Coronaviruse” abbreviated CoV refers to a family of enveloped, positive-sense, single-stranded, and highly diverse RNA viruses, with four distinct groups (i.e. alpha, beta, gamma, and delta). The α-coronavirus and β-coronavirus are of particular interest, because of their ability to cross from non-human animal to humans. So far, there are seven documented human coronaviruses (hCoVs), including the beta-genera CoVs, namely Severe Acute Respiratory Syndrome (SARS)—CoV (SARS-CoV), Middle East Respiratory Syndrome (MERS)—CoV (MERS-CoV), SARS-CoV hCoV-HKU1, and hCoV-OC43 and the α-genera CoVs, which are hCoV-NL63 and hCoV-229E, respectively.

Any terms not directly defined herein shall be understood to have the meanings commonly associated with them as understood within the art of the invention.

Various alternative embodiments and examples are described herein. These embodiments and examples are illustrative and should not be construed as limiting the scope of the invention.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way.

Various alternative embodiments and examples are described herein. These embodiments and examples are illustrative and should not be construed as limiting the scope of the invention.

### Materials and Methods

Cell culture. Caco-2 cells (ATCC HTB-37™), Calu-3 cells (ATCC HTB-55™) and Vero E6 cells (ATCC CRL-1586™) were cultivated in accordance with ATCC recommendations. Human hepatoma, Huh-7.5.1 cells [44] were obtained from APATH LLC and cultivated in Dulbecco's modified Eagle's medium (DMEM) supplemented with 10% fetal bovine serum, 0.1 mM nonessential amino acids, 1 mM sodium pyruvate, and 10 mM HEPES. Experiments were performed in Huh-7.5.1 and Vero E6 cells below passage 30, and Caco-2 and Calu-3 cells below passage 6. All cells were expanded in T75 flask with a with 5% carbon dioxide at 37° C. Cell density was kept between 0.25 and 2 million cells/mL.

Intestinal biopsy-derived colonoids from healthy donors were donated from the Johns Hopkins Conte Digestive Disease Basic and Translational Research Core Center (NIH NIDDK P30-DK089502) and grown based on methods by Staab et al. [45].

Compounds. Compounds were solubilized in 100% dimethyl sulfoxide (DMSO, Sigma™) in a concentration of 10 mM and stored at −20° C. Compounds were diluted in cell-specific media prior to treatment, limited to less than 5 freeze-thaw cycles for each aliquot. Compounds were obtained from Takeda Pharmaceutical Co.™[49] in a GSK3β-focused library, which are also described in Takeda Pharmaceutical Company™ patent US20100069381 (WO2008016123).

Viral Infection Assays. All SARS-CoV-2 infections were carried out in a Biological Contamination Level 3 (BCL3) facility (UBC FINDER) in accordance with Public Health Agency of Canada and UBC FINDER regulations. SARS-CoV-2 SB2 was isolated by Dr. Samira Mubareka (Sunnybrook Hospital, ON, Canada) [46] and was passaged in Vero E6 cells. For experiments, passage 3 of the virus was used with a determined viral titer of 1.5×10⁷ plaque forming units (PFU)/mL. B.1.617.2 (delta) and BA.1 (omicron) variants obtained from BEI Resources (ATCC) and passaged in Vero E6 cells. HCoV-229E was kindly obtained from Dr. Eric Jan, (UBC, BC, Canada) and infections were carried out in Biological Contamination Level 2+ laboratory in Huh-7.5.1 cells (HCoV-229E) at 33° C. Cells were seeded at a concentration of 10,000 cells/well in 96-well plates, 24 h prior to infection. SARS-CoV-2 stocks were diluted in cell-specific media to a Multiplicity of Infection (MOI) of 1 for Caco-2, Vero E6, and Huh-7.5.1, and MOI of 2 for Calu-3 cells and colonoids. Cells were pre-treated with compounds for 3 hours and then incubated with the virus for 2 days (except for colonoids, which incubated for 3 days), followed by fixation of the cells with 3.7% formalin for 30 minutes for immunostaining and to inactivate the virus. The fixative was removed, and washed with PBS, followed by immunostaining of the cells with the mouse primary antibody J2 (dsRNA; Jena Biosciences™) and rabbit primary antibody HL344 (SARS-CoV-2 nucleocapsid; GeneTex™) at working dilutions of 1:1000 for 1 h at room temperature. Secondary antibodies were used at a 1:2000 dilution and included the goat-anti mouse IgG Alexa Fluor 488™ (cat #A11001) and goat-anti rabbit IgG Alexa Fluor 555™ (A21428) from Invitrogen™ with the nuclear stain Hoechst 33342™ at 1 μg/mL for 1 h in room temperature in the dark. After antibody removal, plates were kept covered in aluminum foil until scanning to avoid photobleaching.

KAM-2000 array analysis. Huh-7.5.1 cells seeded at 5×10⁶ cells in T175 flasks were infected with SARS-CoV-2 at an MOI of 1 as described above. Samples were prepared for the KAM-2000 antibody array with chemical cleavage at cysteine (CCC) residues according to the manufacturer's recommendations (Kinexus Bioinformatics Corp.™). Briefly, after 48 h of infection, infected cells were washed twice with PBS then harvested by scraping in 100 μL of Lysis buffer (5 mM EDTA, 2 mM EGTA, 20 mM MOPS, 50 mM NaF, 25 mM Na4P207, 2.5 mM Na3VO4, 60 mM beta-glycerophosphate, 50 nM Phenylarsine oxide, 1% Triton X-100, Protease Inhibitor Cocktail, 5 μM Pepstatin A, 0.5 mM Tris(2carboxyethyl)phosphine hydrochloride, pH9) provided by the manufacturer (Kinexus Bioinformatics Corp.™). 2-nitro-5-thiocyanatobenzoic acid was added afterwards to the samples at a final concentration of 6 mM. After 15 min of incubation at 37° C., samples were centrifuged at 50 000 rpm for 30 min at 18° C. Supernatants were collected and protein concentrations quantified. Samples were further processed by Kinexus Bioinformatics™.

High content screening methodology and parameters. Monitoring of the total number of cells (based on nuclei staining) and number of virus infected cells (based on dsRNA and Nucleocapsid staining) was performed using the CellInsight CX7™ High Content Screening platform (Thermo Fisher™), as previously described (33042061). Shortly, nuclei are identified and counted using the 350′461 nm wavelength (Hoechst 33342™); cell debris and other particles are deducted based on a size filter tool. A Region of Interest (ROI, or “circle”) is then drawn around each host cell and validated against the bright field image to correspond with host cell membranes. The ROI encompasses the “spots”, where dsRNA (485/521 nm wavelength) and SARS-CoV-2 nucleocapsid (533/588 nm wavelength) are localized. Finally, the software (HCS Studio Cell Analysis Software™, version 4.0) identifies, counts and measures the pixel area and intensity of the spots within each circle. The fluorescent spot area measured within each cell (circle) is then added and quantified for each well. The total circle spot intensity of each well corresponds to the viral load, and is normalized to non-infected cells, and to cells infected with no compound. Nuclei stain (Hoechst 33342™) was used to quantify cell loss (due to cytotoxicity or loss of adherence), and to normalize the changes in viral load resulting from a decrease in cell numbers.

Confocal microscopy. Huh 7.5.1 cells were seeded at 100,000/slide in a 24-well plate and cultured in DMEM+10% FBS for 24 h. They were subsequently infected with SARS CoV-2 at MOI of 2 for 1 h in a 5% carbon dioxide incubator at 37° C. The inoculum removed and replaced with complete media containing 1% DMSO or 10 μM GSK3β inhibitor. Uninfected cells received only complete media. Cells were left to grow for another 2 days, washed with cold PBS and fixed with 4% paraformaldehyde for 30 mins. The fixative was removed, cells washed with PBS, and permeabilized with 0.1% Triton x100 for 4 min. Cells were again washed with PBS and blocked with 1% BSA for 1 h. BSA was removed and cells were incubated with the primary antibodies mouse monoclonal antibody to S1 protein (GTX635708, 1:500) and rabbit polyclonal antibody to nucleocapsid protein (GTX635679, 1:1000) for 1 h at room temperature. Secondary antibodies were used at a 1:1000 dilution, incubated for 1 h with Fluoroshield DAPI™ (Abcam™, Ab 104139) for nuclear staining, removed, washed with PBS, and mounted in aqueous mounting medium. Images were captured on the Zeiss Axio Observer Z1™ inverted fluorescent microscope (Zeiss™, Germany). The fluorescent intensity was calculated using ImageJ Software™.

Dose-response analysis. Intracellular dose-response of Viral Load (dsRNA or Nucleocapsid signal) in the presence of compounds was performed at dilution factors of 1:1 with 3 technical replicates in each experiment, and at least 3 biological replications per concentration of compound. Viral Loads were interpolated to negative control (0.1% DMSO, no infection)=0, and positive control (0.1% DMSO, with infection)=100. GraphPad Prism 9™ (GraphPad Software, Inc.™) non-linear regression fit modeling variable slope was used to generate a dose-response curve (Y=Bottom+(Top-Bottom)/(1+10\hat{ }((LogIC50-X)*HillSlope)), constrained to top=100, bottom=0.

Sources of recombinant proteins, peptides and antibodies. The following recombinant SARS-CoV-2 proteins were expressed in E. coli and procured from the MRC Protein Phosphorylation and Ubiquitination Unit Reagents and Services at the University of Dundee (Dundee, Scotland): myelin basic protein-spike receptor binding domain (MBP-Spike RBD) amino acids (aa) 319-541 (DU 67753), glutathione S-transferase-nonstructural protein-1 (GST-NSP1) aa 1-180 (DU 66413), GST-NSP2 aa 1-638 (DU 66414); NSP13 aa 1-601 (DU 66417), GST-NSP14 aa 1-527 (DU 66418), GST-NSP15 aa 1-527 (DU 66419), GST-membrane protein aa 1-222 (DU 67699), and GST-nucleocapsid protein aa 1-419 (DU 67726). Additional recombinant SARS-CoV-2 proteins were produced as described [35]. This included fusion proteins with a carrier protein module, a thermophilic family 9 carbohydrate-binding module 208 (CBM9).

All of the protein kinases used in this study were active preparations of recombinant, GST-fusion human proteins expressed in E. coli or Sf9 insect cells and sourced from SignalChem™ (Richmond, BC, Canada). The following kinases were tested: cyclin-dependent kinase 2/cyclin A2 (CDK2/A2; C29-10G), casein kinase 1-alpha-1 (CK1a1; C64-10G), casein kinase 1-delta (CK161; C65-10G), casein kinase 1-gamma-1 (CK1γ1; C68-11G), casein kinase 2-alpha-1 (CK2a1; C70-10G), extracellularly-regulated kinase-1 (ERK1; M29-10G), glycogen synthase kinase-3-beta (GSK3p; Go9-10G), cAMP-dependent protein kinase catalytic subunit-alpha (PKACa; P51-10G), and protein kinase C-alpha (PKC α; P61-18G). Production of all synthetic peptides modeled after the sequences in the SARS-CoV-2 proteins was performed by Lifetein™ (Somerset, NJ, USA).

Peptide affinity-purified rabbit polyclonal antibodies directed against synthetic peptides were acquired from Kinexus Bioinformatics™ and based on the following SARS-CoV-2 proteins: nucleocapsid aa 156-170 (NNCOV2N-1), ORF1a aa 735-750—NSP2 (NNCOV2-1A-2), and spike aa 574-588 (NNCOV2S-10).

Protein kinase reactions. All protein kinase reactions were performed with the ADP-Glo Kinase Assay from ProMega™ (Madison, WI, USA). Substrate peptides were assayed at a final concentration of ˜250 μM with 250 μM ATP for 30 min at 30° C. in a final volume of 25 μL. Recombinant nucleocapsid was similarly assayed at a final concentration of 13.4 ng/μL. The concentrations of recombinant protein kinases used were ˜ 2 ng/μL.

Western blotting. To investigate protein expression levels, infected cells were lysed by scraping in the following lysis buffer: 20 mM 3-(N-morpholino) propanesulfonic acid (MOPS), pH 7.2, 5 mM EDTA, 2 mM ethylene glycol tetraacetic acid (EGTA), 0.5% (vol/vol) Triton X-100, 30 mM NaF, 20 mM Na₄P₂O₇, 1 mM Na₃VO₄, 40 mM β-glycerophosphate, 1 mM dithiothreitol (DTT), 1 mM phenylmethylsulfonyl fluoride, 3 mM benzamidine, 5 μM pepstatin A, and 10 μM leupeptin and 0.5 μM Aprotonin, 0.5 mM Tris (2-carboxyethyl) phosphine hydrochloride (TCEP)-pH-9.0. Cells were briefly sonicated and 2-Nitro-5-thiocyanatobenzoic acid (NTCB) was added to a final concentration of 6 mM. Tubes were rotated several times to thoroughly expose the contents to lysis buffer and ensure virus inactivation, and samples were subsequently frozen at −20° C. Similarly-treated infected cells were tested for plaque formation using a standard PFU test to confirm the lysis buffer deactivated viral particles prior to removal of the assay tubes from BCL3. The tubes were incubated in a 37° C. water bath for 15 min and then subjected to ultracentrifugation at 100,000×g for 30 mins. The resulting samples were resolved by SDS-PAGE and transferred onto a nitrocellulose membrane. The blot was probed with the designated antibodies and developed by enhanced chemiluminescence.

Statistical analysis and artwork. Statistical analysis were performed and figures were prepared using GraphPad Prism 9™ (GraphPad Software, Inc.). Imbedded blots and fluorescent images were generated using GIMP™.

### EXAMPLES

### Example 1: Screening of GSK30 Library

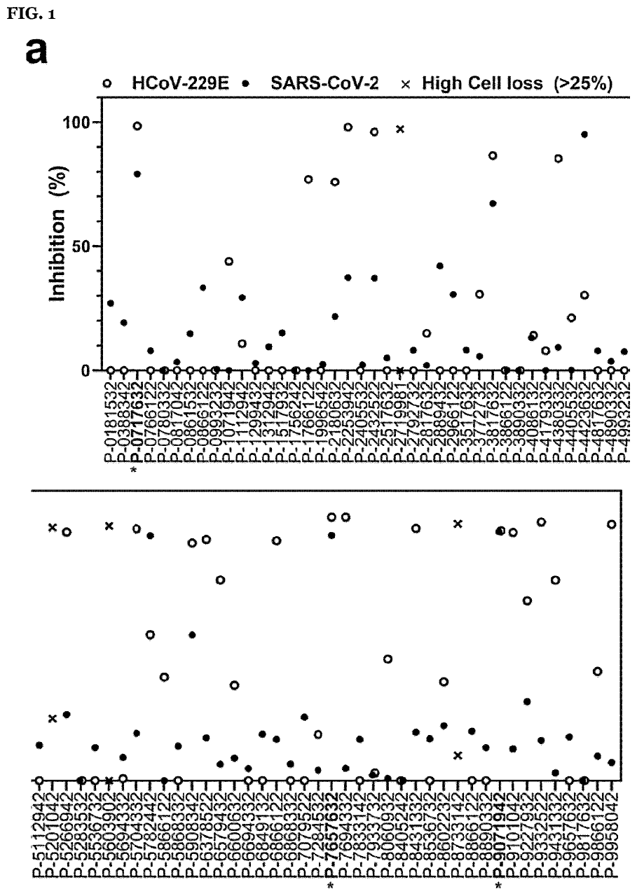

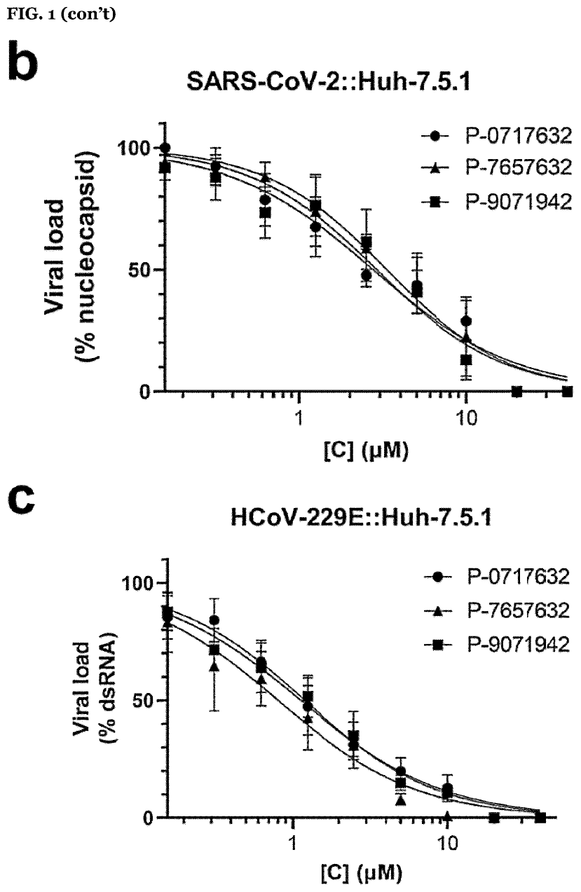

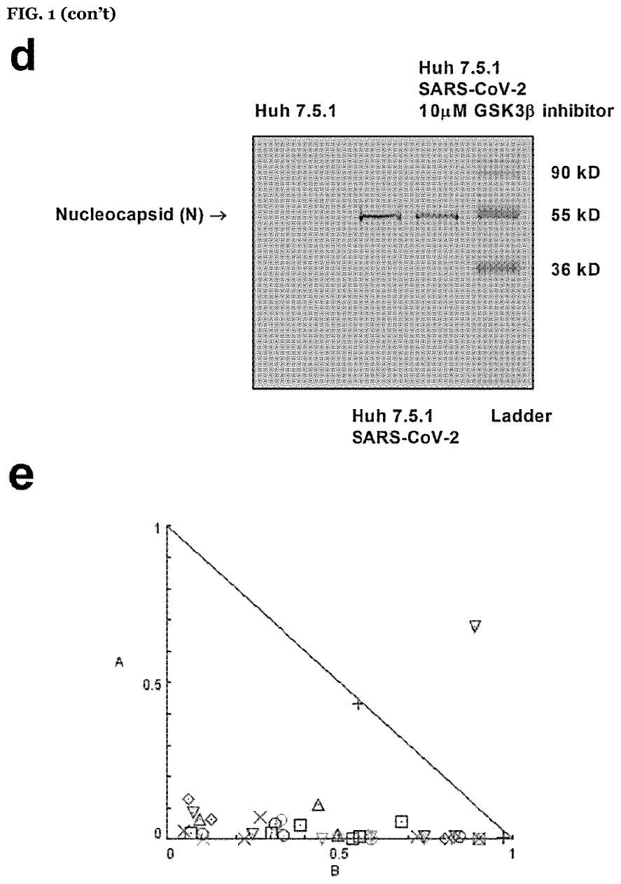

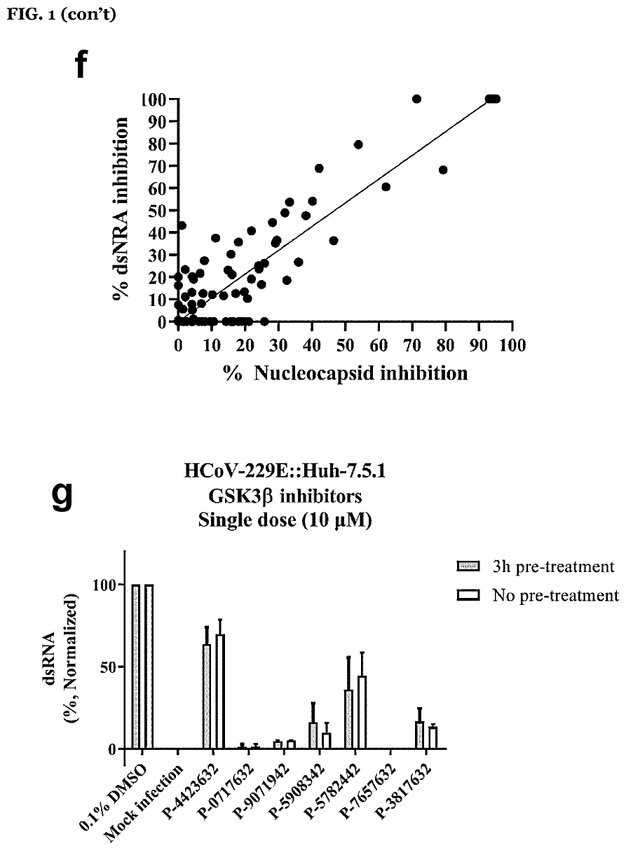

Despite successful vaccine development, drugs against SARS-CoV-2 are still needed to manage active infections. Building on existing knowledge regarding the involvement GSK3β, we aimed at identification of compounds active against this key human kinase as potential SARS-CoV-2 inhibitors. A focused GSK3β inhibitor library [18] and described in U.S. Pat. No. 8,492,378, provided by Takeda Pharmaceutical Company™ (Japan) was screened against both SARS-CoV-2 and human alpha coronavirus HCoV-229E-infected Huh-7.5.1 cells and monitored for either dsRNA or N protein levels as markers of infection (TABLE A). As seen in FIG. 1a, a high proportion of compounds active against GSK3β was effective at 10 μM at reducing SARS-CoV-2 and HCoV-229E infection. In measuring N protein, a marker of viral translation, of the 83 screened compounds, 7 compounds (8%) showed 50% inhibition, with nearly half (n=40; 48%) of the compounds having some inhibitory effect (>1000) (FIG. 1a). Furthermore, we probed for the presence of dsRNA, a marker of viral replication in many RNA viruses. Screen inhibition of N protein and dsRNA yielded similar readouts, with a mean difference of 1000, values differing by less than 1SD of the average in the majority (8300) of comparisons and a regression slope of 1.067×, which showed inhibition of N protein transcription is a reliable indicator of replication inhibition and of general viral load in this assay.

TABLE A
Glycogen Synthase Kinase-3-beta (GSK3β) Inhibitor Library Screen
Inhibition of glycogen synthase kinase-3-beta (GSK3β) blocks
nucleocapsid phosphorylation and SARS-CoV-2 and replication

| | | | 0.7 | 0.5 | | |
|---|---|---|---|---|---|---|
| | 0.7 | 0.3 | SARS- | SARS- | Average | |
| | HCoV- | SARS- | CoV-2 | CoV-2 | SARS- | |
| | 229E | CoV- | inhi- | inhibi- | CoV-2 | Aver- |
| | inhi- | 2 inhi- | bition (% | tion (% | Nucleo- | age |
| | bition | bition | Nucleo- | Nucleo- | capsid | cell |
| Z′ | (% | (% | capsid) | capsid) | inhi- | loss |
| Takeda ID | dsRNA) | dsRNA) | 1 | 2 | bition | (%) |
| P-0181532 | 0.0 | 47.6 | 50.0 | 26.4 | 38.2 | 15.5 |
| P-0388342 | 0.0 | 23.1 | 21.7 | 8.1 | 14.9 | 20.7 |
| P-0717632 | 97.6 | 68.2 | 75.2 | 83.3 | 79.3 | 24.4 |
| P-0766122 | 0.0 | 30.3 | 11.7 | 20.0 | 15.8 | 34.1 |
| P-0780332 | 0.0 | 0.6 | 0.0 | 0.0 | 0.0 | 9.2 |
| P-0817042 | 0.0 | 0.0 | 0.0 | 31.3 | 15.7 | 0.0 |
| P-0861532 | 0.0 | 36.5 | 18.5 | 40.5 | 29.5 | 25.0 |
| P-0866122 | 0.0 | 48.9 | 46.7 | 17.2 | 31.9 | 32.1 |
| P-0993232 | 0.0 | 0.0 | 0.0 | 2.1 | 1.0 | 13.6 |
| P-1071942 | 43.9 | 0.0 | 0.0 | 0.0 | 0.0 | 15.7 |
| P-1112942 | 10.8 | 0.0 | 9.5 | 28.5 | 19.0 | 9.2 |
| P-1299432 | 0.0 | 0.0 | 0.0 | 21.1 | 10.6 | 9.9 |
| P-1312942 | 0.0 | 0.0 | 0.0 | 0.0 | 0.0 | 22.5 |
| P-1517932 | 0.0 | 26.2 | 13.9 | 37.8 | 25.8 | 16.7 |
| P-1756242 | 0.0 | 20.1 | 0.0 | 0.0 | 0.0 | 13.9 |
| P-1766122 | 77.0 | 0.0 | 0.0 | 3.4 | 1.7 | 27.7 |
| P-1996542 | 0.0 | 0.0 | 0.0 | 0.0 | 0.0 | 0.0 |
| P-2180632 | 75.9 | 19.0 | 9.2 | 0.0 | 4.6 | 33.0 |
| P-2253942 | 98.0 | 68.9 | 47.8 | 36.7 | 42.2 | 25.3 |
| P-2405532 | 0.0 | 0.0 | 0.0 | 0.0 | 0.0 | 5.8 |
| P-2432522 | 96.1 | 36.4 | 35.8 | 57.3 | 46.5 | 21.0 |
| P-2517632 | 0.0 | 23.4 | 4.1 | 0.0 | 2.1 | 3.7 |
| P-2719981 | 0.0 | 0.0 | 0.0 | 0.0 | 0.0 | 64.3 |
| P-2792732 | 0.0 | 5.1 | 0.0 | 8.5 | 4.2 | 17.7 |
| P-2817632 | 14.9 | 21.1 | 0.0 | 32.3 | 16.1 | 8.5 |
| P-2889432 | 0.0 | 79.5 | 61.2 | 46.6 | 53.9 | 20.3 |
| P-2966122 | 0.0 | 19.1 | 0.6 | 43.2 | 21.9 | 23.0 |
| P-3517632 | 0.0 | 0.0 | 5.2 | 27.7 | 16.5 | 10.9 |
| P-3772732 | 30.7 | 13.1 | 0.0 | 8.3 | 4.1 | 36.0 |
| P-3817632 | 87.7 | 100.0 | 70.8 | 72.0 | 71.4 | 12.7 |
| P-3866122 | 0.0 | 11.1 | 0.0 | 4.2 | 2.1 | 40.1 |
| P-3890332 | 0.0 | 7.5 | 0.0 | 0.0 | 0.0 | 9.6 |
| P-4086132 | 14.1 | 27.4 | 4.4 | 11.2 | 7.8 | 8.4 |
| P-4179332 | 7.9 | 0.0 | 0.0 | 18.8 | 9.4 | 12.3 |
| P-4380332 | 85.4 | 23.7 | 0.0 | 48.4 | 24.2 | 13.8 |
| P-4405532 | 21.2 | 0.0 | 0.0 | 39.9 | 19.9 | 14.8 |
| P-4423632 | 43.2 | 100.0 | 98.0 | 92.4 | 95.2 | 12.0 |
| P-4817632 | 0.0 | 43.2 | 1.9 | 0.0 | 1.0 | 8.3 |
| P-4890332 | 0.0 | 37.5 | 22.4 | 0.0 | 11.2 | 17.8 |
| P-4993232 | 0.0 | 1.1 | 0.0 | 9.1 | 4.5 | 0.0 |
| P-5112942 | 0.0 | 13.4 | 19.8 | 19.7 | 19.8 | 32.1 |
| P-5201042 | 0.0 | 10.4 | 0.0 | 41.4 | 20.7 | 29.0 |
| P-5266942 | 94.3 | 0.0 | 0.0 | 51.6 | 25.8 | 14.0 |
| P-5283532 | 0.0 | 16.3 | 0.0 | 0.0 | 0.0 | 0.0 |
| P-5536732 | 0.0 | 35.7 | 23.5 | 12.4 | 18.0 | 4.0 |
| P-5603902 | 0.0 | 0.0 | 0.0 | 15.9 | 8.0 | 60.8 |
| P-5694332 | 0.8 | 0.0 | 0.0 | 12.2 | 6.1 | 8.8 |
| P-5704332 | 95.6 | 26.7 | 17.8 | 54.3 | 36.0 | 8.2 |
| P-5782442 | 60.0 | 100.0 | 93.3 | 94.4 | 93.8 | 16.1 |
| P-5817632 | 39.3 | 24.4 | 0.0 | 47.9 | 24.0 | 2.4 |
| P-5866122 | 0.0 | 12.6 | 0.0 | 14.8 | 7.4 | 34.4 |
| P-5868332 | 12.3 | 8.0 | 12.6 | 1.1 | 6.9 | 14.9 |
| P-5908342 | 91.5 | 60.5 | 76.0 | 48.4 | 62.2 | 21.7 |
| P-6378522 | 76.2 | 18.5 | 21.1 | 43.8 | 32.5 | 11.3 |
| P-6579432 | 36.2 | 11.6 | 0.0 | 27.2 | 13.6 | 10.5 |
| P-6600632 | 0.0 | 12.6 | 20.5 | 14.0 | 17.2 | 12.3 |
| P-6694332 | 0.0 | 21.6 | 0.0 | 13.0 | 6.5 | 12.1 |
| P-6849132 | 91.1 | 35.2 | 7.5 | 50.8 | 29.1 | 23.9 |
| P-6866122 | 0.0 | 44.5 | 27.9 | 28.4 | 28.2 | 41.8 |
| P-6868332 | 0.0 | 0.0 | 0.0 | 0.0 | 0.0 | 18.7 |
| P-7079522 | 17.5 | 0.0 | 8.1 | 24.5 | 16.3 | 19.7 |
| P-7284532 | 36.8 | 0.0 | 0.0 | 0.0 | 0.0 | 7.5 |
| P-7657632 | 100.0 | 100.0 | 100.0 | 86.2 | 93.1 | 25.4 |
| P-7694332 | 0.0 | 0.0 | 0.0 | 0.0 | 0.0 | 9.1 |
| P-7833142 | 2.9 | 0.0 | 0.0 | 28.6 | 14.3 | 31.9 |
| P-7933732 | 46.2 | 20.3 | 6.7 | 1.7 | 4.2 | 9.2 |
| P-8060932 | 0.0 | 12.1 | 0.0 | 20.4 | 10.2 | 26.7 |
| P-8405242 | 95.7 | 5.5 | 0.0 | 2.9 | 1.4 | 24.9 |
| P-8431332 | 0.0 | 0.0 | 14.2 | 0.0 | 7.1 | 14.5 |
| P-8536732 | 37.6 | 0.0 | 0.7 | 7.1 | 3.9 | 13.6 |
| P-8602232 | 0.0 | 0.0 | 0.0 | 5.0 | 2.5 | 27.1 |
| P-8733142 | 0.0 | 16.0 | 0.0 | 0.0 | 0.0 | 45.6 |
| P-8866122 | 0.0 | 16.6 | 0.0 | 49.8 | 24.9 | 33.9 |
| P-8890332 | 33.7 | 0.0 | 12.8 | 23.3 | 18.0 | 17.1 |
| P-9071942 | 94.2 | 100.0 | 100.0 | 89.1 | 94.5 | 29.9 |
| P-9101042 | 68.3 | 25.1 | 34.7 | 13.5 | 24.1 | 7.8 |
| P-9227932 | 98.2 | 54.1 | 47.3 | 33.1 | 40.2 | 16.6 |
| P-9332522 | 76.1 | 0.0 | 0.0 | 42.2 | 21.1 | 28.0 |
| P-9431332 | 0.0 | 7.8 | 8.2 | 0.0 | 4.1 | 5.5 |
| P-9657632 | 0.0 | 53.7 | 23.0 | 43.6 | 33.3 | 33.9 |
| P-9817632 | 41.5 | 0.0 | 0.0 | 8.6 | 4.3 | 17.8 |
| P-9866122 | 97.3 | 40.8 | 0.0 | 43.7 | 21.9 | 12.4 |
| P-9958042 | 0.0 | 0.0 | 2.1 | 14.1 | 8.1 | 15.3 |

Screening for inhibition of N protein and dsRNA yielded similar readouts (FIG. 1f), indicating that relative N protein translation abundance may be used as a reliable proxy for infection and viral load. Probing of dsRNA is an oft-used method, as it serves as a specific marker of viral replication in RNA viruses. Using the dsRNA marker (FIG. 1A), screening against HCoV-229E and SARS-CoV-2 demonstrated that targeting GSK3β resulted in effective viral control in 31% and 19% of the compounds (≥50% infection inhibition), respectively. Together, the high ratio of potential hits and relatively low toxicity of the library as a whole support the viability of GSK3β as a target for HDT in COVID-19 and other human coronaviruses infections. Compared to the ‘hit’ rate of non-targeted host-directed therapy screens of ˜3% [25, 26] or ˜0.1% in non-specific screens [27], our results demonstrate a high benefit-to-investment ratio. More importantly, this screen demonstrated that more general host-kinase screens, which typically have 1-4 compounds representing each host kinase type, might be insufficient to rule-out kinases as potential targets. This is made evident through our study, as many GSK3β inhibitors in this library did not pass the 50% activity threshold. As such, a larger, more comprehensive focused screen might be a better approach compared to general representative screens. Although beyond the scope of this study, targeted libraries also offer fascinating opportunities for further structure-function relationship studies.

### Example 2: Dose and Cell Dependent Analysis of Hit Compound

Three active compounds, P-0717632 (T-1686568), P-7657632, and P-9071942, were chosen for their high activity against both viruses, were tested for their ability to reduce SARS-CoV-2 and HCoV-229E viral infection. Dose-response assays showed that ED₅₀ values were ˜3 μM and not significantly different between the selected inhibitors (FIGS. 1b and 1c). Pre-treatment of cells with the three compounds did not appear to improve antiviral activity (FIG. 1g). Selectivity Index (SI) was additionally measured, as a value of cytotoxic concentration (CC₅₀) over ED₅₀. Toxicity of the compounds against Huh 7.5.1 host cells was determined: P-0717632 was the most tolerated at a CC₅₀>100 μM (SARS-CoV-2 Selectivity Index SI>36), while P-7657632 and P-9071942 CC₅₀ values were at 39 μM (SARS-CoV-2 SI=14) and 27 μM (SARS-CoV-2 SI=8), respectively (TABLE 1). P-0717632's favourable SI value made it a suitable candidate for further analysis; it was validated to reduce SARS-CoV-2 N protein translation in whole cells extract (FIG. 1d) and it was shown to act synergistically with the FDA-approved antiviral Remdesivir in HCoV-229E-infected cells. Using the Chou-Talalay method [19], an average Combination Index (CI) value of 0.7 (FIG. 1e) was determined. CI values<1 are defined as synergistic, CI=1 as additive, and CI>1 as antagonistic.

TABLE 1
Exemplary Compounds with Activity and one compound without Activity

| Compound | CAS No. | Activity | |
|---|---|---|---|
| Identifier | (Takeda Ex. [49]) | (SARS-CoV-2) | Structure |
| P-0717632 (T1686568) | 1005204-23-8 (416) | ED₅₀ = 2.7 μM CC₅₀ > 100 μM SI = 68 | 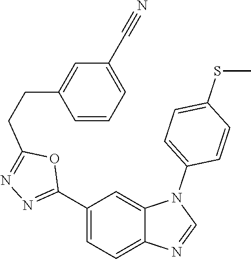 [SMILES: CSc1ccc(-n2cnc3ccc(-c4nnc(CCc5cccc(C#N)c5)o4)cc32)cc1] |
| P-9071942 (T-17610197) | 1005203-77-9 (370) | ED₅₀ = 3.3 μM CC₅₀ = 27 μM SI = 8 | 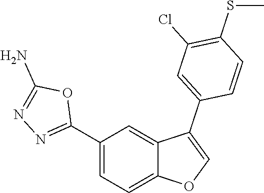 [SMILES: CSc1ccc(-c2coc3ccc(-c4nnc(N)o4)cc23)cc1Cl] |
| P-7657632 (T-1686955) | 1005202-65-2 (259) | ED₅₀ = 2.9 μM CC₅₀ = 39 μM SI = 14 | 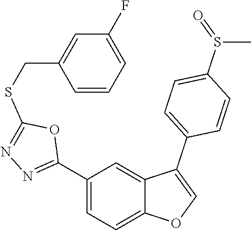 [SMILES: CS(=O)c1ccc(-c2coc3ccc(-c4nnc(SCc5cccc(F)c5)o4)cc23)cc1] |
| P-5782442 (T-1761263) | | active (see TABLE A) | 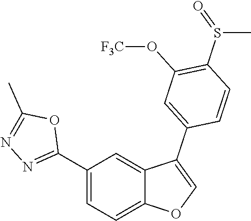 [SMILES: Cc1nnc(-c2ccc3occ(-c4ccc(S(C)=O)c(OC(F)(F)F)c4)c3c2)o1] |
| P-3817632 (T-1686571) | | active (see TABLE A) | 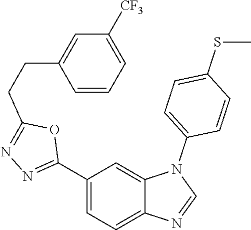 [SMILES: CSc1ccc(-n2cnc3ccc(-c4nnc(CCc5cccc(C(F)(F)F)c5)o4)cc32)cc1] |
| P-4423632 | | active (see TABLE A) | 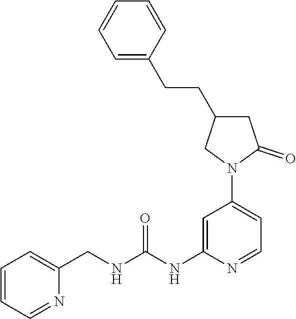 [SMILES: O=C(NCc1ccccn1)Nc1cc(N2CC(CCc3ccccc3)CC2=O)ccn1] |
| P-0780332 | | inactive (see TABLE A) | 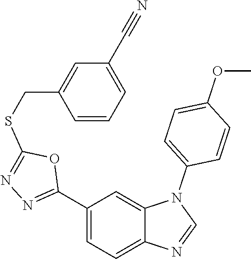 [SMILES: COc1ccc(-n2cnc3ccc(-c4nnc(SCc5cccc(C#N)c5)o4)cc32)cc1] |

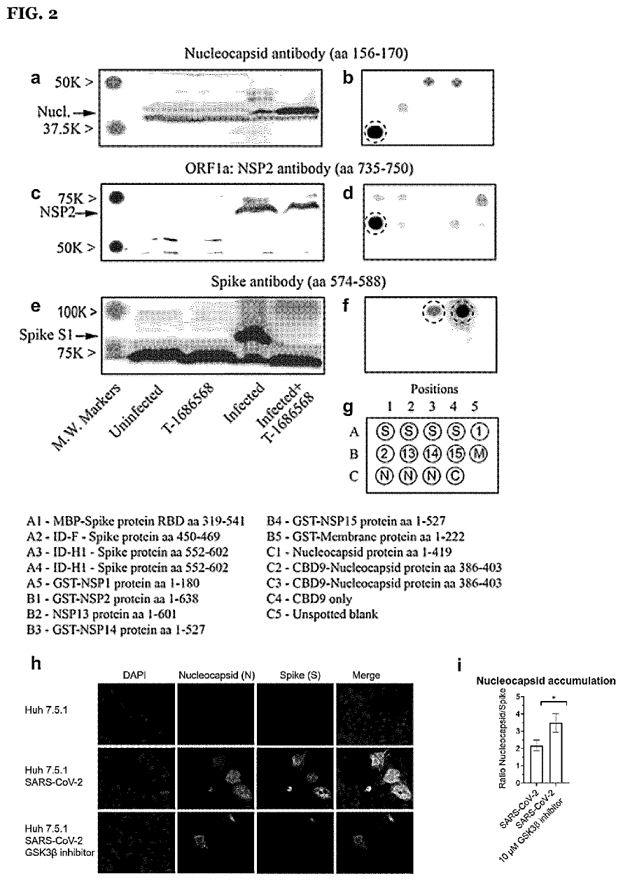

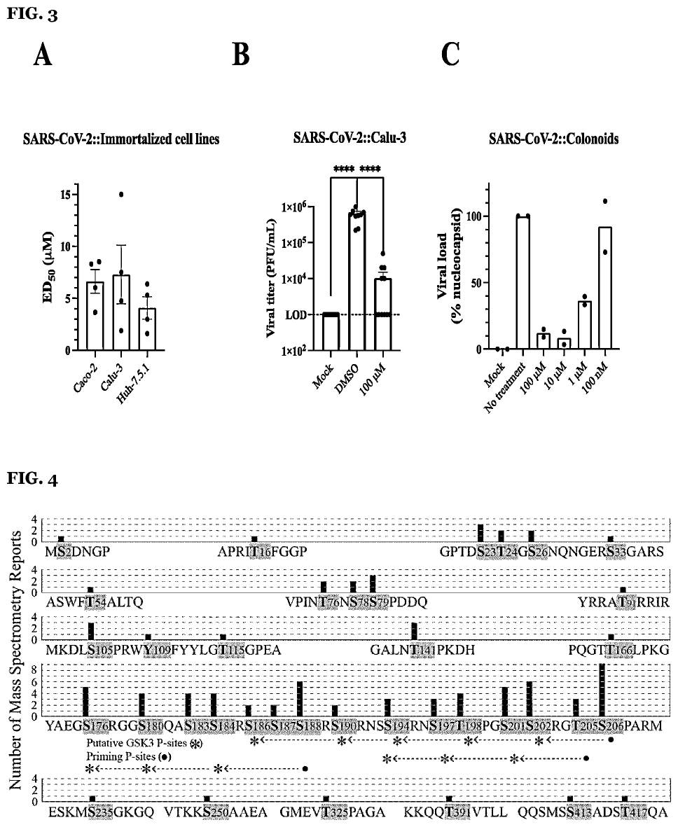

Relative inhibitor activity is often influenced by variability in different cell infection models [20]. While liver-derived Huh-7.5.1 cells were suitable for screening, initial validation, and toxicity, we performed a further investigation Of P-0717632 potency in two other common SARS-COV-2 infection models, colon Caco-2 cells and the clinically-relevant lung Calu-3 cells. In all investigated cell lines, P-0717632 provided a similar robust activity, with ED₅₀ ranging from 4 to 7 μM (FIG. 3a). This observation indicates that GSK43P activity is needed for SARS-COV-2 replication, independent of host cell type. These results demonstrated a reduction of intracellular viral markers; however, this may not directly translate to a reduction in viral production and release. We therefore measured the effect of P-0717632 on the release of infective particles from SARS-CoV-2 infected Calu-3 cells and observed a significant 2-log reduction 48 hours after infection (FIG. 3b). In addition to the WT SARS-CoV-2 strain, P-0717632 reduced intracellular viral titres in the B.1.617.2 and BA.1 variants (FIG. 1g).

To approach a patient model for SARS-CoV-2, donor-derived organoids or induced pluripotent stem cell-derived organoids are used as the closest analogs. These organoids can be infected with SARS-CoV-2, have greater cell variability than monoclonal immortalized cell lines, and do not carry tumorigenic artefacts [21, 22]. Using donor intestinal organoids monolayer infected with SARS-CoV-2, P-0717632 also showed a proxy efficacy in a dose-dependent manner to greatly decrease intracellular N protein levels (FIG. 3c). Western blot to detect SARS-CoV-2 proteins showed Spike S1 protein expression in infected Huh-7.5.1 cells was blocked by treatment with P-0717632, yet the non-structural protein (NSP2) and N protein could still be detected as shown in FIGS. 3a-f.

Comparative analysis of SARS-CoV-2 and HCoV-229E screens suggests that GSK3β inhibition is not limited to SARS-CoV [48] and SARS-CoV-2, may serve as a general coronavirus protection acting against the common nucleocapsid targets shared between multiple human coronaviruses species. Considering the timeline for new drug development and approval is lengthy and continued coronavirus spread is likely, the ongoing COVID-19 pandemic necessitates exploratory development of such broad-spectrum antivirals. This is further supported by conserved arginine-serine rich domains, target regions for GSK3 phosphorylation, in N protein sequences across coronaviruses. Despite limited 20-30% overall N protein sequence similarity, the domains maintain repeat motifs conducive for repeat phosphorylation [7].

### Example 3: P-0717632 Treatment Results in Population Reduction in Viral Markers, and in Accumulation of Viral N Protein in Remaining Infected Cells

The Spike (S) protein of SARS-CoV-2 is another important outcome of viral translation, and one that is not known to be phosphorylated directly by GSK3, so can be used as a reference for viral load. S protein expression in infected Huh-7.5.1 cells was reduced by treatment with ED₈₀ P-0717632 (10 μM), yet the non-structural protein (NSP2) and N protein could still be detected, with an apparent accumulation of the N protein (FIG. 2). The specificity of the antibodies used to monitor the levels of the S, NSP2 and N proteins was validated with dot blots with recombinant versions of these and other SARS-CoV-2 proteins and peptides (FIG. 2). Fluorescence microscopy confirmed overall population reduction in N protein and S protein levels were reduced upon treatment with ED₈₀ P-0717632 (FIG. 2h), yet importantly N protein was accumulated in remaining infected cells (FIG. 2i). The treatment effect was extended to reduction of cellular syncytia formation and filopodial protrusions (FIG. 2h) previously shown to be associated with SARS CoV-2 infection [23, 24].

### Example 4: GSK3D Phosphorylates Primed S180/S184, S190/S194 and T198/S202 Sites of the SARS-CoV-2 N Protein

Bioinformatic in silico and experimental data from multiple mass spectrometry studies identified 37 phosphorylation sites in the N protein from coronaviruses (FIG. 4). At least 15 of these confirmed phosphorylation sites were located at Ser-176 to Ser-276 within the arginine-serine-rich (RS) domain, with many complementing consensus phosphosite recognition sequences in GSK3 [20]. Using in vitro kinase assays, nine different recombinant host kinases were used to phosphorylate a set of SARS-CoV-2 phosphopeptides, synthetic nucleocapsid peptides modeled after eight phosphosites in the RS domain. Half of these peptides were artificially pre-phosphorylated at sites that were predicted to be subsequently secondarily phosphorylated by GSK3. GSK3β further phosphorylated all four of these phosphopeptides and protein kinase C-alpha (PKCα) was observed to be able to phosphorylate three of the four pairs of peptides provided that they were not already pre-phosphorylated at the GSK3 priming phosphosites (TABLE 2). Interestingly, this in vitro phosphorylation assays showed that GSK3β phosphorylates more effectively peptides containing S180/S184, S190/S194 and T198/S202, which have already been primed (i.e. phosphorylated) in the adjacent phospho-sites S188, T198 and S206, respectively. Both GSK3β and PKC α phosphorylate the full nucleocapsid (TABLE 3). Indeed, Western blot with phosphosite-specific antibodies, confirmed S180/S184, S190/S194 and T198 phosphorylation sites in SARS-CoV-2 nucleocapsid that was already primed in the residues S188, T198 and S206 respectively. These assays showed that both GSK3β and PKCα phosphorylate these peptides to varying degrees, but unexpectedly the combination of the two did not yield overall higher phosphorylation activity. This is likely due to incomplete priming of the peptides by PKCα in the limited duration of the phosphotransferase assays (TABLE 3).

TABLE 2
In vitro phosphorylation of synthetic peptides modeled after SARS-CoV-2
nucleocapsid protein phosphorylation sites by protein kinases using the ADP-Glo method.
Relative phosphorylation is shown in comparison to the signal recorded as Relative Light Units
(RLU) with each protein kinase incubated in the absence of added peptide substrate with ATP
alone. Values are averages from duplicate measurements. NT = not tested. Predicted GSK3
phosphorylation sites are bolded and underlined.

| | | Relative Phosphorylation to | | | | | | | | |
|---|---|---|---|---|---|---|---|---|---|---|
| | | Autophosphorylation Control (set at 100) | | | | | | | | |
| | Peptide | CDK2 + | | | | | | | | |
| Peptide | Sequence | Cyclin | | | | | | | | |
| Name | (SEQ ID NO:) | A2 | CK1a1 | CK1d | CK1g1 | CK2a1 | ERK1 | GSK3b | PKACa | PKCa |
| SCV2-N | RGGSQASSRSS | NT | NT | NT | NT | NT | NT | 1209 | 128 | 389 |
| [177-190]- | (pS)RS (7) | | | | | | | | | |
| ps188 | | | | | | | | | | |
| SCV2-N | RGGSQASSRSSSR | NT | NT | NT | NT | NT | NT | 107 | 162 | 1320 |
| [177-190] | S (8) | | | | | | | | | |
| SCV2-N | SQASSRSSSR(pS) | NT | NT | NT | NT | 100 | NT | 111 | 312 | 418 |
| [180-193]- | RNS (5) | | | | | | | | | |
| pS190 | | | | | | | | | | |
| SCV2-N | SQASSRSSSRSRN | NT | NT | NT | NT | 101 | NT | 102 | 123 | 1278 |
| [180-193] | S (6) | | | | | | | | | |
| SCV2-N | SSRSRNSSRNS(pT) | 101 | 250 | 152 | NT | 102 | 100 | 1042 | 157 | 553 |
| [187-200]- | PG (3) | | | | | | | | | |
| pT198 | | | | | | | | | | |
| SCV2-N | SSRSRNSSRNSTP | 100 | 145 | 100 | NT | 103 | 100 | 108 | 485 | 1080 |
| [187-200] | G (4) | | | | | | | | | |
| SCV2-N | RNSTPGSSRGT(pS) | 100 | NT | 105 | 105 | NT | 100 | 1068 | 205 | NT |
| [195-209]- | PAR (1) | | | | | | | | | |
| pS206 | | | | | | | | | | |
| SCV2-N | RNSTPGSSRGTSP | 104 | NT | 101 | 104 | NT | 102 | 137 | 151 | NT |
| [195-209] | AR (2) | | | | | | | | | |
| Nucleocapsid | Full length | 100 | 99 | NT | 102 | 103 | 100 | 101 | 110 | 101 |
| protein | | | | | | | | | | |

| | | | | | | | | | |
|---|---|---|---|---|---|---|---|---|---|
| Autophosphorylation | 5335 | 558 | 514 | 6175 | 2772 | 5636 | 3035 | 136 | 2459 |
| signal (RLU) | | | | | | | | | |

TABLE 3
In vitro phosphorylation of synthetic peptides modeled after SARS-CoV-2
nucleocapsid protein phosphorylation sites by recombinant protein kinase C-alpha and
glycogen synthase kinase-beta using the ADP-Glo method. Relative phosphorylation is
shown in comparison to the signal with the nucleocapsid protein incubated in the absence
of added substrate with ATP alone. NT = not tested. Predicted GSK3 phosphorylation
sites are bolded and underlined.

| | | Relative Phosphorylation to No | | |
|---|---|---|---|---|
| | | Kinase Control | | |
| | Peptide Sequence | Mean ± S.Dev. (n = 3) | | |
| Peptide | (SEQ ID NO: ) | PKCa | GSK3b | PKCa + GSK3b |
| SCV2-N [177-190]- | RGGSQASSRSS(pS)RS | 3861 ± 71 | 24,585 ± 645 | NT |
| pS188 | (7) | | | |
| SCV2-N [177-190] | RGGSQASSRSSSRS | 13,714 ± 502 | 2161 ± 63 | 3572 ± 22 |
| | (8) | | | |
| SCV2-N [180-193]- | SQASSRSSSR(pS)RNS | 4161 ± 66 | 2957 ± 3 | NT |
| pS190 | (5) | | | |
| SCV2-N [180-193] | SQASSRSSSRSRNS | 16,366 ± 261 | 2407 ± 23 | 3836 ± 41 |
| | (6) | | | |
| SCV2-N [187-200]- | SSRSRNSSRNS(pT)PG | 7032 ± 319 | 23,031 ± 486 | NT |
| pT198 | (3) | | | |
| SCV2-N [187-200] | SSRSRNSSRNSTPG | 12,687 ± 486 | 2573 ± 49 | 3692 ± 61 |
| | (4) | | | |
| SCV2-N [195-209]- | RNSTPGSSRGT(pS)PAR | 1531 ± 47 | 24,082 ± 381 | NT |
| ps206 | (1) | | | |
| SCV2-N [195-209] | RNSTPGSSRGTSPAR | 3301 ± 114 | 3291 ± 57 | 4262 ± 27 |
| | (2) | | | |
| Nucleocapsid | Full length | 1032 ± 9 | 2052 ± 90 | 2754 ± 66 |
| protein | | | | |
| No peptide | Autophosphorylation | 1282 ± 51 | 1995 ± 133 | 2897 ± 38 |
| substrate | | | | |
| Nucleocapsid | No kinase control | 100 ± 6 | | |
| protein | | | | |

Using SARS-CoV-2 nucleocapsid as a measurement of viral load resulted in a robust Z′ score for the screen (Z′=0.6) when compared alternatively to the dsRNA marker (Z′=0.3, not shown), likely due to ability of the antiviral dsRNA antibody to cross react with the host cell dsRNA. One viral marker may be insufficient to draw conclusions regarding viral load, as in the case of GSK3β, due to potential target-marker interactions. Although treated cells do accumulate nucleocapsid, we found a strong agreement between nucleocapsid and dsRNA markers levels in their response to the inhibitors tested, suggesting that in this assay, nucleocapsids can be used as a marker for viral load. Screen inhibition readouts were similar (mean Δ of 10%) to those in nucleocapsid expression, as shown in TABLE 2. Media-released virus using PFU quantification confirmed that the inhibitory effect of GSK3β-inhibition extends to SARS-CoV-2 assembly and maturation.

TABLE 4
Sequences

| SEQ | |
|---|---|
| ID NO. | Sequence |
| 1 | RNSTPGSSRGT(pS)PAR |
| 2 | RNSTPGSSRGTSPAR |
| 3 | SSRSRNSSRNS(pT)PG |
| 4 | SSRSRNSSRNSTPG |
| 5 | SQASSRSSSR(pS)RNS |
| 6 | SQASSRSSSRSRNS |
| 7 | RGGSQASSRSS(pS)RS |
| 8 | RGGSQASSRSSSRS |
| 9 | MSDNGP |
| 10 | APRITFGGP |
| 11 | GPTDSTGSNQNGERSGARS |
| 12 | ASWFTALTQ |
| 13 | VPINTNSSPDDQ |
| 14 | YRRATRRIR |
| 15 | MKDLSPRWYFYYLGTGPEA |
| 16 | GALNTPKDH |
| 17 | PQGTTLPKG |
| 18 | YAEGSRGGSQASSRSSSRSRNSSRNSTPGSSRGTSPARM |
| 19 | ESKMSGKGQVTKKSAAEAGMEVTPAGAKKQQTVTLLQQSM |
| | SSADSTQA |

### Example 4: Validation of Anti SARS-CoV-2 Activity in Different Cell Lines

P-0717632 was selected due to its high selectivity index. Similar to the validation of viral load measurements using different viral markers, utilizing different cell lines is important for any robust immortalized cell line screen, and particularly for compound activity against SARS-CoV-2, given the many reporting mismatches of screen results between different cell lines [20]. To guard against this, we internally validated our screen through the use of different cell lines and found a consistent P-0717632 inhibitory effect. Of particular note, host-targeting inhibitors designed against viral entry mechanisms, such as ACE2 and TMPRSS2, present higher variability in the expression of these host factors between cell lines [28]. Our observations suggest GSK3β is a conserved pathway critical to SARS-CoV-2 infection in many tissues, and strongly recommend it be considered in the selection of targets for drug development. This is further supported through the observation of SARS-CoV-2 inhibition in patient-derived colon organoids, which contain multiple different cell types that may be infected.

Despite successful vaccine development, drugs against SARS-CoV-2 are still needed to manage active infections. Building on existing knowledge regarding the involvement of GSK3β in infection progression, we aimed to identify compounds active against this key human kinase as potential coronaviridae inhibitors. Screening of a targeted library yielded multiple hits of active compounds with greater than 50% inhibition of viral infection, nearly half of which conferred some inhibitory effect against SARS-CoV-2 and HCoV-229E. Compared to the ˜3% ‘hit’ rate of non-targeted host-directed therapy screens [25] [26] or−0.1% in non-specific screens [27], our results demonstrate a high benefit-to-investment ratio.

The efficacy of GSK3β inhibition strategy against two different coronavirus strains in these screens demonstrates that this strategy is not limited to SARS-CoV[6] and SARS-CoV-2, and thus may be an appropriate target for future drug development against various Coronaviridae. This is further supported by the presence of conserved RS domains as the target regions for GSK3 phosphorylation in N protein sequences across coronaviruses [7]. In light of the ongoing coronavirus transmission and protracted drug development and approval timelines, the urgency of the COVID-19 pandemic necessitates exploration of such broad-spectrum antivirals.

A singular viral marker may be insufficient to accurately reflect viral infection load, particularly if the marker has potential target-marker interaction, as in the case of GSK3β and the N protein. The SARS-CoV-2 nucleocapsid served as a reliable measurement of viral infection, resulting in a robust Z′ score for the screen (Z′=0.6). The dsRNA marker showed slightly reduced robustness (Z′=0.3, TABLE A), likely due to the cross-reactivity of the antiviral dsRNA antibody to that of the host cell. We found a strong agreement between nucleocapsid and dsRNA levels during the screen, indicating that despite being a target for post-translational modifications by GSK3, the nucleocapsid can be used as a viral infection marker in this type of assay. Relative screen inhibition readouts were similar (mean difference of 10%) to those of nucleocapsid expression. Reductions of the spike protein levels, and media-released virus, measured using plaque assay quantification, confirmed that the effect of GSK3β inhibition extends to SARS-CoV-2 assembly and maturation.

Given reported screen result inconsistencies of compound activity against SARS-CoV-2 across different cell lines [20], including different cell lines is important for any robust screening campaign. To guard against this, we internally validated our screen through the use of different cell lines and found a consistent P-0717632 inhibitory effect. While some host-targeting inhibitors designed for viral entry surface molecules, such as against ACE2 and TMPRSS2, present higher expression variability between cell lines [28], our observations indicate GSK3β is a conserved pathway critical to SARS-CoV-2 infection in many tissues and thus is a strong candidate target for drug development. The broad applicability of targeting GSK3β is further supported through observed SARS-CoV-2 inhibition in patient-derived colon organoids, which contain multiple cell types that are susceptible to infection [29].

Mass spectrometry studies have revealed that the N protein's RS domain at residues Ser-176 to Ser-276 undergoes extensive phosphorylation (FIG. 4). Using synthetic peptides modeled after these phosphosites, we have shown they are directly phosphorylated by recombinant GSK3β, provided these peptides are pre-phosphorylated at priming sites. Recombinant PKCα was able to phosphorylate several of the priming sites for GSK3β in vitro, although other kinases may be responsible for this step in vivo. Our findings complement the recent in vivo studies of Liu et al., [7] which showed that GSK3 acted upstream of the N protein to mediate its phosphorylation. Through site-directed mutagenesis, they further determined that the Ser-188 and Ser-206 priming phosphosites for recognition by GSK3 were required for phosphorylation-dependent mobility shifts of the N protein. Additionally, Thr-205 may act a priming site for Ser-201 and Ser-197 phosphorylation, a process potentially controlled by GSK3 activity.

The phosphorylation of the RS domain in SARS-CoV and SARS-CoV-2 has been implicated in the regulation of N protein binding to RNA, multimerization and subcellular location [30-34]. Studies with SARS-CoV-2 indicate that changes in the phosphorylation status of the RS domain induces profound alterations in the association of multiple nucleocapsid proteins with a single viral RNA in a structured oligomer with RNA-protein and protein-protein interactions to switch to one that permits more viral genome processing [35]. Following N protein phosphorylation in the RS domain, the RNA-protein complex is able to recruit the stress granule protein G3BP1 and suppress the G3BP-dependent host immune response [36]. In our study, the blockage of N protein phosphorylation by GSK3 appeared to result in partial accumulation of the N protein, and prevention of spike protein production to allow formation of the virus particles.

Other recent studies [35, 37-42] have demonstrated that the N protein also undergoes liquid-liquid phase separation when mixed with RNA; it is predicted through polymer theory that the same multivalent interactions driving phase separation also enable RNA compaction.

Phosphorylation also plays a role in spike protein behaviour [43], potentially affecting expression levels and cellular trafficking. Our results support this relationship as lower levels of the spike protein (FIG. 2) matched P-0717632-induced viral titer reduction. Our collective findings across multiple assays strongly demonstrate GSK3β inhibition may serve as an effective antiviral strategy for early intervention in COVID-19 and other coronavirus infections.

The disclosure may be further understood by the non-limiting examples. Although the description herein contains many specific examples, these should not be construed as limiting the scope of the disclosure but as merely providing illustrations of some of the embodiments of the disclosure. For example, thus the scope of the disclosure should be determined by the appended aspects and their equivalents, rather than by the examples given.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this disclosure for a given application. In specific applications, the selection of a given anion or cation for preparation of a salt may result in increased or decreased solubility of that salt. Every formulation or combination of components described or exemplified herein may be used to practice the disclosure, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings. Unless otherwise explained, all technical and scientific terms used herein have the same meaning as commonly understood by one of ordinary skill in the art to which a disclosed disclosure belongs.

### REFERENCES

1. John Hopkins Corona Viruse Resource Center. 2021; Available from: https://coronavirus.jhu.edu/map.html.
2. Chen, J., et al., Epidemiological and economic impact of COVID-19 in the US. Sci Rep, 2021. 11(1): p. 20451.
3. Sen, P., et al., Burden and characteristics of COVID-19 in the United States during 2020.
Nature, 2021. 598(7880): p. 338-341.
4. Gordon, D. E., et al., A SARS-CoV-2 protein interaction map reveals targets for drug repurposing. Nature, 2020. 583(7816): p. 459-468.
5. Coleman, C. M., et al., Abelson Kinase Inhibitors Are Potent Inhibitors of Severe Acute Respiratory Syndrome Coronavirus and Middle East Respiratory Syndrome Coronavirus Fusion. J Virol, 2016. 90(19): p. 8924-33.
6. Wu, C. H., et al., Glycogen synthase kinase-3 regulates the phosphorylation of severe acute respiratory syndrome coronavirus nucleocapsid protein and viral replication. J Biol Chem, 2009. 284(8): p. 5229-39.
7. Liu, X., et al., Targeting the coronavirus nucleocapsid protein through GSK-3 inhibition. Proc Natl Acad Sci USA, 2021. 118(42).
8. Rana, A. K., et al., Glycogen synthase kinase-3: A putative target to combat severe acute respiratory syndrome coronavirus 2 (SARS-CoV-2) pandemic. Cytokine Growth Factor Rev, 2021. 58: p. 92-101.
9. Chang, C. K., et al., Modular organization of SARS coronavirus nucleocapsid protein. J Biomed Sci, 2006. 13(1): p. 59-72.
10. Embi, N., D. B. Rylatt, and P. Cohen, Glycogen synthase kinase-3 from rabbit skeletal muscle. Separation from cyclic-AMP-dependent protein kinase and phosphorylase kinase. Eur J Biochem, 1980. 107(2): p. 519-27.
11. Tung, H. Y., et al., The catalytic subunits of protein phosphatase-1 and protein phosphatase 2A are distinct gene products. Eur J Biochem, 1984. 138(3): p. 635-41.
12. Goode, N., et al., Differential regulation of glycogen synthase kinase-3 beta by protein kinase C isotypes. J Biol Chem, 1992. 267(24): p. 16878-82.
13. Barrell, W. B., H. L. Szabo-Rogers, and K. J. Liu, Novel reporter alleles ofGSK-3alpha and GSK-3beta. PLoS One, 2012. 7(11): p. e50422.
14. Plyte, S. E., et al., Glycogen synthase kinase-3: functions in oncogenesis and development. Biochim Biophys Acta, 1992. 1114(2-3): p. 147-62.
15. Cohen, P. and S. Frame, The renaissance of GSK3. Nat Rev Mol Cell Biol, 2001. 2(10): p. 769-76.
16. Saraswati, A. P., et al., Glycogen synthase kinase-3 and its inhibitors: Potential targetfor various therapeutic conditions. Eur J Med Chem, 2018. 144: p. 843-858.
17. Wang, H., et al., GSK3beta and the control of infectious bacterial diseases. Trends Microbiol, 2014. 22(4): p. 208-17.
18. Saitoh, M., et al., Design, synthesis and structure-activity relationships of 1,3,4-oxadiazole derivatives as novel inhibitors of glycogen synthase kinase-3beta. Bioorg Med Chem, 2009. 17(5): p. 2017-29.
19. Chou, T. C. and P. Talalay, Quantitative analysis of dose-effect relationships: the combined effects ofmultiple drugs or enzyme inhibitors. Adv Enzyme Regul, 1984. 22: p. 27-55.
20. Kumar, S., et al., Clinically relevant cell culture models and their significance in isolation, pathogenesis, vaccine development, repurposing and screening of new drugs for SARS-CoV-2: a systematic review. Tissue Cell, 2021. 70: p. 101497.
21. Mulay, A., et al., SARS-CoV-2 infection of primary human lung epitheliumfor COVID-19 modeling and drug discovery. bioRxiv, 2020.
22. Han, Y., et al., Identification of SARS-CoV-2 inhibitors using lung and colonic organoids.

Nature, 2021. 589(7841): p. 270-275.
23. Braga, L., et al., Drugs that inhibit TMEM16 proteins block SARS-CoV-2 spike-induced syncytia. Nature, 2021. 594(7861): p. 88-93.
24. Merolli, A., et al., An intra-cytoplasmic route for SARS-CoV-2 transmission unveiled by Helium-ion microscopy. Sci Rep, 2022. 12(1): p. 3794.
25. Han, H. W., et al., Drug Discovery Platform Targeting M. tuberculosis with Human Embryonic Stem Cell-Derived Macrophages. Stem Cell Reports, 2019. 13(6): p. 980-991.
26. Shapira, T., et al., High-Content Screening of Eukaryotic Kinase Inhibitors Identify CHK2 Inhibitor Activity Against Mycobacterium tuberculosis. Front Microbiol, 2020. 11: p. 553962.
27. Pethe, K., et al., Discovery of Q203, a potent clinical candidate for the treatment of tuberculosis. Nat Med, 2013. 19(9): p. 1157-60.
28. Zecha, J., et al., Data, Reagents, Assays and Merits of Proteomics for SARS-CoV-2 Research and Testing. Mol Cell Proteomics, 2020. 19(9): p. 1503-1522.
29. Shapira, T., et al., A novel highly potent inhibitor of TMPRSS2-like proteases blocks SARS-CoV-2 variants of concern and is broadly protective against infection and mortality in mice. bioRxiv, 2021.
30. Nelson, G. W., S. A. Stohlman, and S. M. Tahara, High affinity interaction between nucleocapsid protein and leader/intergenic sequence of mouse hepatitis virus RNA. J Gen Virol, 2000. 81(Pt 1): p. 181-8.
31. Chen, H., et al., Mass spectroscopic characterization of the coronavirus infectious bronchitis virus nucleoprotein and elucidation of the role of phosphorylation in RNA binding by using surface plasmon resonance. J Virol, 2005. 79(2): p. 1164-79.
32. Peng, T. Y., K. R. Lee, and W. Y. Tarn, Phosphorylation of the arginine/serine dipeptide-rich motif of the severe acute respiratory syndrome coronavirus nucleocapsid protein modulates its multimerization, translation inhibitory activity and cellular localization. FEBS J, 2008. 275(16): p. 4152-63.
33. Stohlman, S. A., et al., Synthesis and subcellular localization of the murine coronavirus nucleocapsid protein. Virology, 1983. 130(2): p. 527-32.
34. Surjit, M., et al., The severe acute respiratory syndrome coronavirus nucleocapsid protein is phosphorylated and localizes in the cytoplasm by 14-3-3-mediated translocation. J Virol, 2005. 79(17): p. 11476-86.
35. Carlson, C. R., et al., Phosphoregulation of Phase Separation by the SARS-CoV-2 N Protein Suggests a Biophysical Basis for its Dual Functions. Mol Cell, 2020. 806): p. 1092-1103 e4.
36. Lu, S., et al., The SARS-CoV-2 nucleocapsid phosphoprotein forms mutually exclusive condensates with RNA and the membrane-associated M protein. Nat Commun, 2021. 12(1): p. 502.
37. Cubuk, J., et al., The SARS-CoV-2 nucleocapsid protein is dynamic, disordered, and phase separates with RNA. Nat Commun, 2021. 12(1): p. 1936.
38. Iserman, C., et al., Genomic RNA Elements Drive Phase Separation of the SARS-CoV-2 Nucleocapsid. Mol Cell, 2020. 806): p. 1078-1091 e6.
39. Perdikari, T. M., et al., SARS-CoV-2 nucleocapsid protein phase-separates with RNA and with human hnRNPs. EMBO J, 2020-39(24): p. e106478.
40. Roden, C. A., et al., Double-stranded RNA drives SARS-CoV-2 nucleocapsid protein to undergo phase separation at specific temperatures. bioRxiv, 2021.
41. Savastano, A., et al., Nucleocapsid protein of SARS-CoV-2 phase separates into RNA-rich polymerase-containing condensates. Nat Commun, 2020. 11(1): p. 6041.
42. Wang, S., et al., Targeting liquid-liquid phase separation of SARS-CoV-2 nucleocapsid protein promotes innate antiviral immunity by elevating MAVS activity. Nat Cell Biol, 2021. 23(7): p. 718-732.
43. Ord, M., I. Faustova, and M. Loog, The sequence at Spike S1/S2 site enables cleavage by furin and phospho-regulation in SARS-CoV2 but not in SARS-CoV1 or MERS-CoV. Sci Rep, 2020. 10(1): p. 16944.
44. Zhong, J., et al., Robust hepatitis Cvirus infection in vitro. Proc Natl Acad Sci USA, 2005. 102(26): p. 9294-9.
45. Staab, J. F., et al., Co-Culture System of Human Enteroids/Colonoids with Innate Immune Cells. Curr Protoc Immunol, 2020. 131(1): p. e113.
46. Banerjee, A., et al., Isolation, Sequence, Infectivity, and Replication Kinetics of Severe Acute Respiratory Syndrome Coronavirus 2. Emerg Infect Dis, 2020. 26(9): p. 2054-2063.
47. Alfhili, M. A., et al., GSK-3-associated signaling is crucial to virus infection of cells. Biochim Biophys Acta Mol Cell Res, 2020. 1867(10): 118767.
48. Chia-Hsin Wu, et al. Glycogen synthase kinase-3 regulates the phosphorylation of severe acute respiratory syndrome coronavirus nucleocapsid protein and viral replication. J Biol Chem. 2009 Feb. 20; 284(8):5229-39. doi: 10.1074/jbc.M805747200. Epub 2008 Dec. 23.
49. Itoh, F. GSK-3Beta Inhibitor Takeda Pharmaceutical Co. US20100069381 (WO2008016123).
50. Berge S. M. et al., Pharmaceutical salts. J. Pharm. Sci. (1977) 66(1):1-19.

1. A compound, the compound having the structure of Formula I: 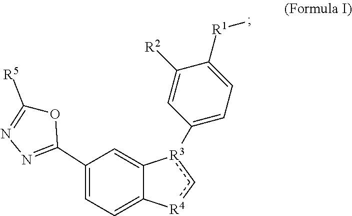
or pharmaceutical acceptable salt or prodrug thereof, for treatment of a viral infection,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁵ is selected from: NH₂; 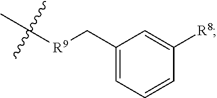
and CH₃;
R⁸ is selected from: CN; halogen; H; CF₃; C₁₋₁₀-alkyl; and OCF₃; and
R⁹ is selected from: CH₂; S; and O.

2. The compound of claim 1, wherein the compound has the structure of Formula II: 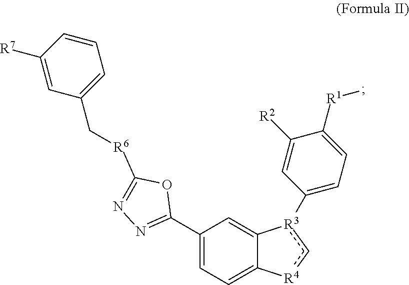
or pharmaceutically acceptable salt or prodrug thereof, for treatment of a viral infection,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁶ is selected from: CH₂; S; and O; and
R⁷ is selected from: CN; halogen; H; CF₃; C₁₋₁₀-alkyl; and OCF₃.

3. The compound of claim 1 or 2, wherein the halogen is selected from F, Cl, Br and I.

4. The compound of claim 1, 2, or 3, wherein the halogen is selected from F and Cl.

5. The compound of any one of claims 1-4, wherein
R¹ is selected from: S; and S═O;
R² is selected from: H; F; Cl; and OCF₃;
R³ is selected from: C; and N;
R⁴ is selected from: O; and N;
R⁵ is selected from: NH₂; 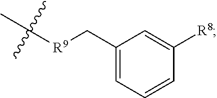
and CH₃;
R⁸ is selected from: CN; F; Cl; H; and CF₃; and
R⁹ is selected from: CH₂; and S.

6. The compound of any one of claims 1-5, wherein
R¹ is selected from: S; and S═O;
R² is selected from: H; Cl; and OCF₃;
R³ is selected from: C; and N;
R⁴ is selected from: O; and N;
R⁵ is selected from: NH₂; 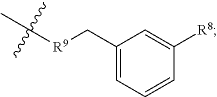
and CH₃;
R⁸ is selected from: CN; F; and CF₃; and
R⁹ is selected from: CH₂; and S.

7. The compound of any one of claims 1-6, wherein the compound is selected from one or more of:

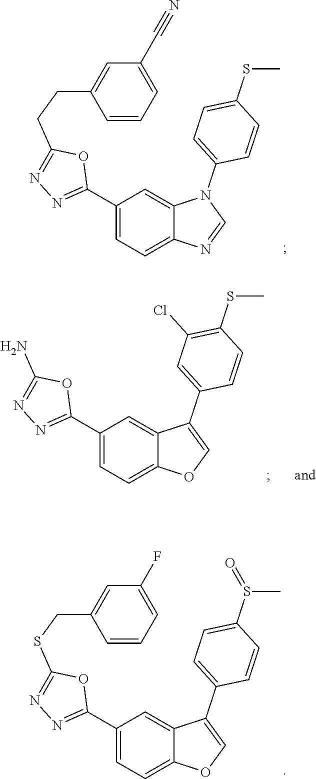 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1]

8. The compound of any one of claims 1-6, wherein the compound is selected from one or more of:

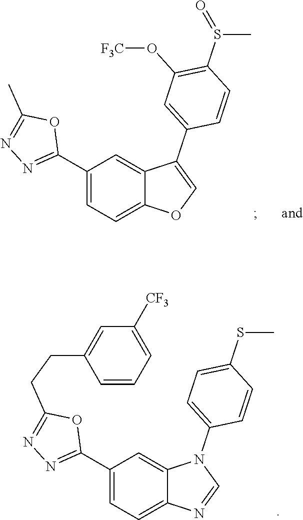 [SMILES: Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

9. The compound of any one of claims 1-6, wherein the compound is selected from one or more of:

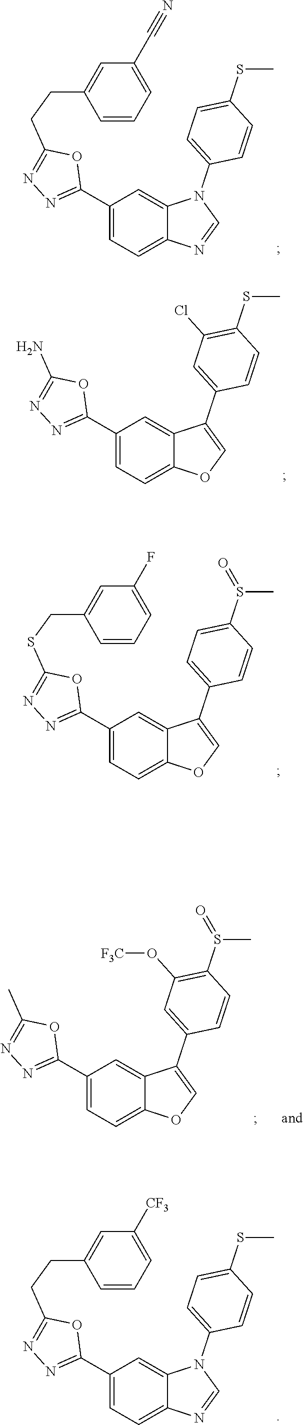 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1 ; Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

10. The compound of any one of claims 1-9, wherein the viral infection is selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection.

11. A method of treating a viral infection in a subject in need thereof, the method comprising administration of a compound to the subject, wherein the compound has the structure of Formula I: 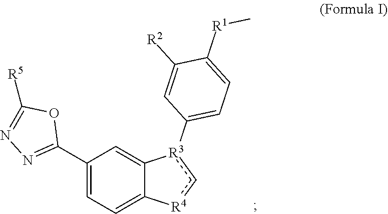
or pharmaceutical acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁵ is selected from: NH₂; 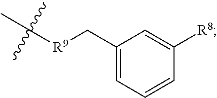
and CH₃;
R⁸ is selected from: CN; halogen; H; CF₃; C₁₋₁₀-alkyl; and OCF₃; and
R⁹ is selected from: CH₂; S; and O.

12. The method of claim 11, wherein the compound has the structure of Formula II: 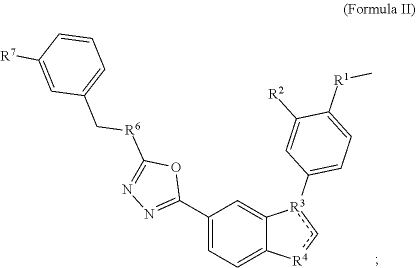
or pharmaceutically acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁶ is selected from: CH₂; S; and O; and
R⁷ is selected from: CN; halogen; H; CF₃; C₁₋₁₀-alkyl; and OCF₃.

13. The method of claim 11 or 12, wherein the halogen is selected from F, Cl, Br and I.

14. The method of claim 11, 12, or 13, wherein the halogen is selected from F and Cl.

15. The method of any one of claims 11-14, wherein
R¹ is selected from: S; and S═O;
R² is selected from: H; F; Cl; and OCF₃;
R³ is selected from: C; and N;
R⁴ is selected from: O; and N;
R⁵ is selected from: NH₂; 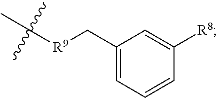
and CH₃;
R⁸ is selected from: CN; F; Cl; H; and CF₃; and
R⁹ is selected from: CH₂; and S.

16. The method of any one of claims 11-15, wherein
R¹ is selected from: S; and S═O;
R² is selected from: H; Cl; and OCF₃;
R³ is selected from: C; and N;
R⁴ is selected from: O; and N;
R⁵ is selected from: NH₂; 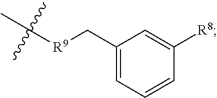
and CH₃;
R⁸ is selected from: CN; F; and CF₃; and
R⁹ is selected from: CH₂; and S.

17. The method of any one of claims 11-16, wherein the compound is selected from one or more of:

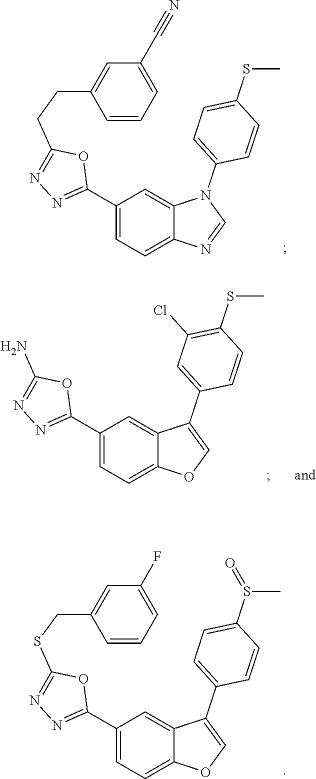 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1]

18. The method of any one of claims 11-16, wherein the compound is selected from one or more of:

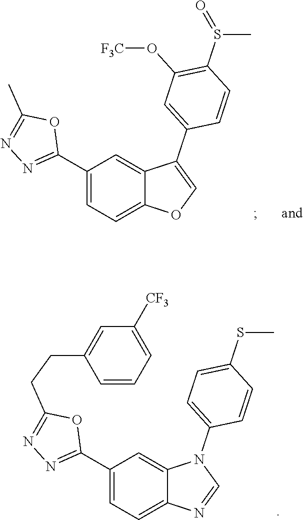 [SMILES: Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

19. The method of any one of claims 11-16, wherein the compound is selected from one or more of:

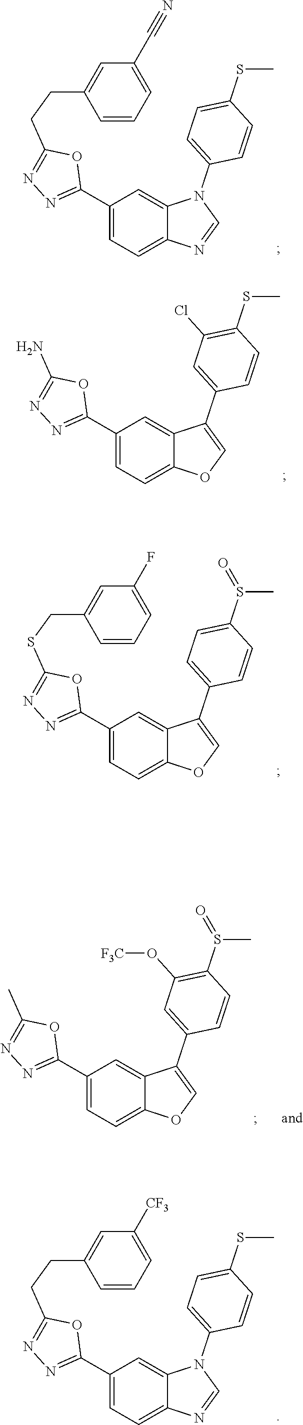 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1 ; Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

20. The method of any one of claims 11-19, wherein the viral infection is selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection.

21. The method of any one of claims 11-20, wherein the viral infection is human coronavirus 229E (HCoV-229E) infection.

22. A pharmaceutical composition, the pharmaceutical composition comprising a compound having the structure of Formula I: 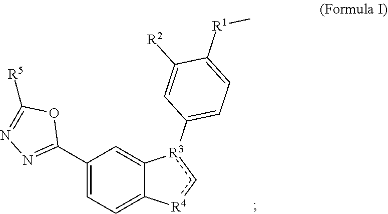
or pharmaceutical acceptable salt or prodrug thereof, for treatment of a viral infection,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁵ is selected from: NH₂; 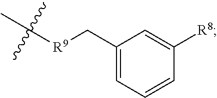
and CH₃;
R⁸ is selected from: CN; halogen; H; CF₃; C₁₋₁₀-alkyl; and OCF₃; and
R⁹ is selected from: CH₂; S; and O.

23. The pharmaceutical composition of claim 22, wherein the compound has the structure of Formula II: 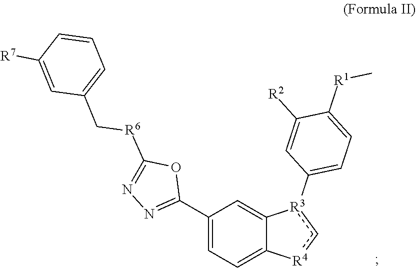
or pharmaceutically acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁶ is selected from: CH₂; S; and O; and
R⁷ is selected from: CN; halogen; H; CF₃; C₁₋₁₀-alkyl; and OCF₃.

24. The pharmaceutical composition of claim 22 or 23, wherein the halogen is selected from F, Cl, Br and I.

25. The pharmaceutical composition of claim 22, 23, or 24, wherein the halogen is selected from F and Cl.

26. The pharmaceutical composition of any one of claims 22-25, wherein
R¹ is selected from: S; and S═O;
R² is selected from: H; F; Cl; and OCF₃;
R³ is selected from: C; and N;
R⁴ is selected from: O; and N;
R⁵ is selected from: NH₂; 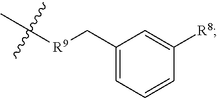
and CH₃;
R⁸ is selected from: CN; F; Cl; H; and CF₃; and
R⁹ is selected from: CH₂; and S.

27. The pharmaceutical composition of any one of claims 22-26, wherein
R¹ is selected from: S; and S═O;
R² is selected from: H; Cl; and OCF₃;
R³ is selected from: C; and N;
R⁴ is selected from: O; and N;
R⁵ is selected from: NH₂; 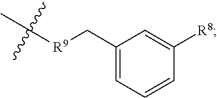
and CH₃;
R⁸ is selected from: CN; F; and CF₃; and
R⁹ is selected from: CH₂; and S.

28. The pharmaceutical composition of any one of claims 22-27, wherein the compound is selected from one or more of:

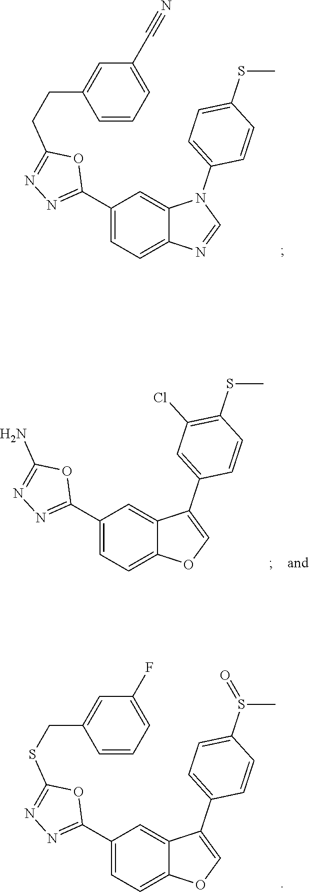 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1]

29. The pharmaceutical composition of any one of claims 22-27, wherein the compound is selected from one or more of:

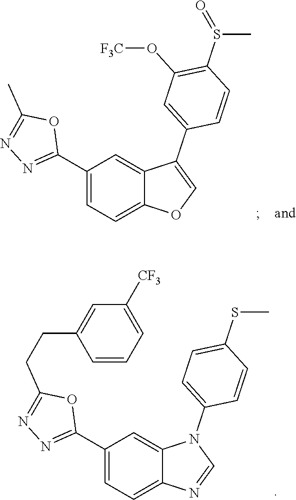 [SMILES: Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

30. The pharmaceutical composition of any one of claims 22-27, wherein the compound is selected from one or more of:

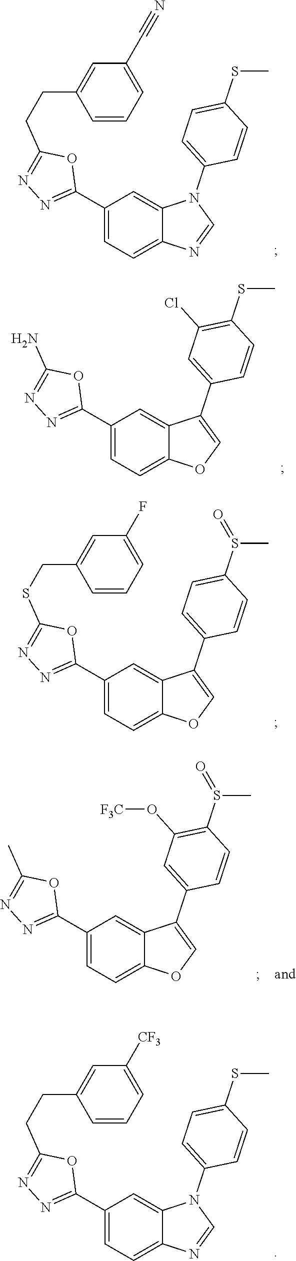

31. The pharmaceutical composition of any one of claims 22-30, wherein the viral infection is selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection.

32. The pharmaceutical composition of any one of claims 22-31, wherein the viral infection is human coronavirus 229E (HCoV-229E) infection.

33. Use of a compound; or a pharmaceutical composition comprising the compound and a pharmaceutically acceptable carrier; for treating a viral infection, the compound having the structure of Formula I: 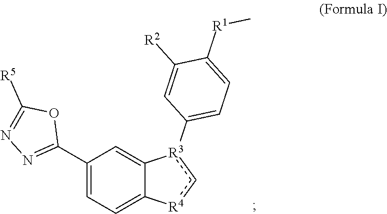
or pharmaceutical acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁵ is selected from: NH₂; 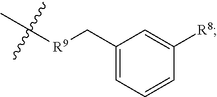
and CH₃;
R⁸ is selected from: CN; halogen; H; CF₃; C₁₋₁₀-alkyl; and OCF₃; and
R⁹ is selected from: CH₂; S; and O.

34. Use of a compound in the manufacture of a medicament for treating a viral infection, the compound having the structure of Formula I: 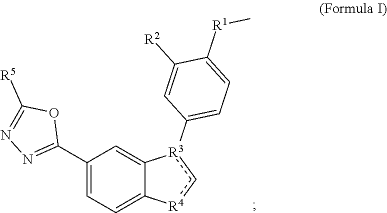
or pharmaceutical acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁵ is selected from: NH₂; 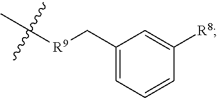
and CH₃;
R⁸ is selected from: CN; halogen; H; CF₃; C₁₋₁₀-alkyl; and OCF₃; and
R⁹ is selected from: CH₂; S; and O.

35. The use of claim 33 or 34, wherein the compound has the structure of Formula II: 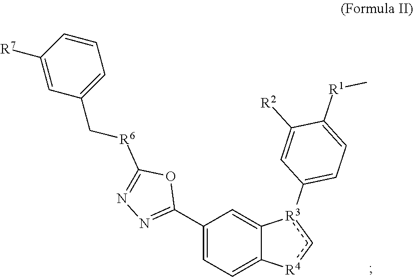
or pharmaceutically acceptable salt or prodrug thereof,
wherein: [symbol]
is optionally a single or a double bond, provided that both are not double bonds and provided that both are not single bonds;
R¹ is selected from: S; S═O; C; and SO₂;
R² is selected from: H; halogen; OCF₃; CF₃; C₁₋₁₀-alkyl; and CN;
R³ is selected from: C; and N;
R⁴ is selected from: O; and N;
R⁶ is selected from: CH₂; S; and O; and
R⁷ is selected from: CN; halogen; H; CF₃; C₁₋₁₀-alkyl; and OCF₃.

36. The use of claim 33, 34, or 35, wherein the halogen is selected from F, Cl, Br and I.

37. The use of any one of claims 33-36, wherein the halogen is selected from F and Cl.

38. The use of any one of claims 33-37, wherein
R¹ is selected from: S; and S═O;
R² is selected from: H; F; Cl; and OCF₃;
R³ is selected from: C; and N;
R⁴ is selected from: O; and N;
R⁵ is selected from: NH₂; 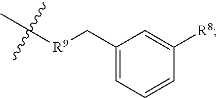
and CH₃;
R⁸ is selected from: CN; F; Cl; H; and CF₃; and
R⁹ is selected from: CH₂; and S.

39. The use of any one of claims 33-38, wherein
R¹ is selected from: S; and S═O;
R² is selected from: H; Cl; and OCF₃;
R³ is selected from: C; and N;
R⁴ is selected from: O; and N;
R⁵ is selected from: NH₂; 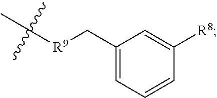
and CH₃;
R⁸ is selected from: CN; F; and CF₃; and
R⁹ is selected from: CH₂; and S.

40. The use of any one of claims 33-39, wherein the compound is selected from one or more of:

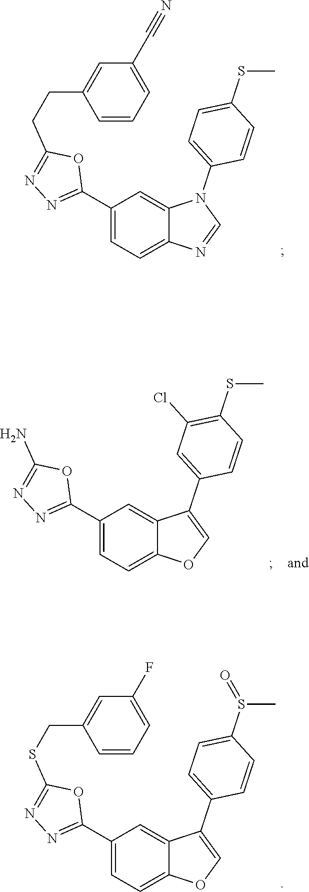 [SMILES: CSc1ccc(-n2cnc3ccc(-c4nnc(CCc5cccc(C#N)c5)o4)cc32)cc1 ; CSc1ccc(-c2coc3ccc(-c4nnc(N)o4)cc23)cc1Cl ; CS(=O)c1ccc(-c2coc3ccc(-c4nnc(SCc5cccc(F)c5)o4)cc23)cc1]

41. The use of any one of claims 33-39, wherein the compound is selected from one or more of:

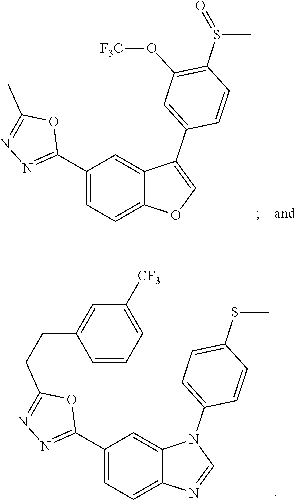 [SMILES: Cc1nnc(-c2ccc3occ(-c4ccc(S(C)=O)c(OC(F)(F)F)c4)c3c2)o1 ; CSc1ccc(-n2cnc3ccc(-c4nnc(CCc5cccc(C(F)(F)F)c5)o4)cc32)cc1]

42. The use of any one of claims 33-39, wherein the compound is selected from one or more of:

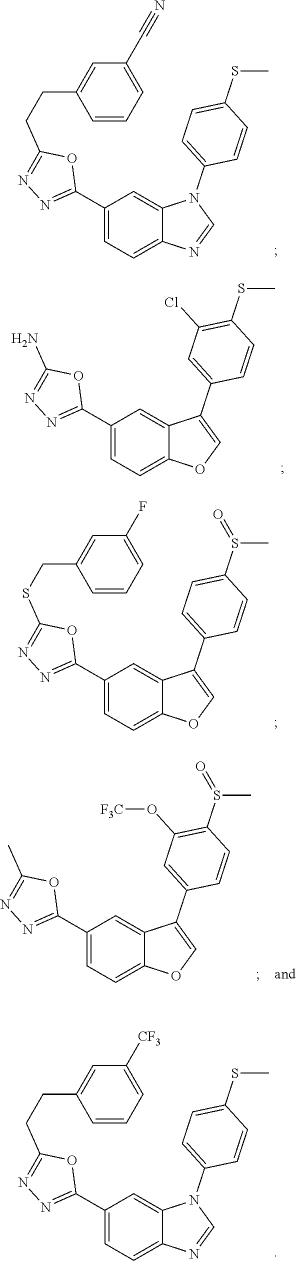

43. The use of any one of claims 33-42, wherein the viral infection is selected from one or more of the following: Severe Acute Respiratory Syndrome (SARS) coronavirus-1 (SARS-CoV-1) infection; SARS coronavirus-2 (SARS-CoV-2) infection; and Middle East Respiratory Syndrome (MERS) coronavirus (MERS-CoV) infection.

44. The use of any one of claims 33-43, wherein the viral infection is a human coronavirus 229E (HCoV-229E) infection.

45. The use of any one of claims 33-43, wherein the treating of the viral infection would benefit from the inhibition of glycogen synthase kinase 3 (GSK3).

46. The use of claim 45, wherein the GSK3 is GSK3β.

47. The use of any one of claims 33-46, wherein the treating of the viral infection would benefit from the inhibition of N protein phosphorylation in a virus.

48. The use of any one of claims 33-46, wherein the treating of the viral infection includes a virus that comprises a nucleocapsid N protein.

49. The use of claim 48, wherein the compound inhibits N protein phosphorylation in the virus.


SEQUENCE LISTING (19 sequences)

SEQ ID NO: 1 - protein, 15 aa, unidentified
  MOD_RES 12: note = PHOSPHORYLATION
RNSTPGSSRGTSPAR

SEQ ID NO: 2 - protein, 15 aa, synthetic construct
RNSTPGSSRGTSPAR

SEQ ID NO: 3 - protein, 14 aa, synthetic construct
  MOD_RES 12: note = PHOSPHORYLATION
SSRSRNSSRNSTPG

SEQ ID NO: 4 - protein, 14 aa, synthetic construct
SSRSRNSSRNSTPG

SEQ ID NO: 5 - protein, 14 aa, synthetic construct
  MOD_RES 11: note = PHOSPHORYLATION
SQASSRSSSRSRNS

SEQ ID NO: 6 - protein, 14 aa, synthetic construct
SQASSRSSSRSRNS

SEQ ID NO: 7 - protein, 14 aa, synthetic construct
  MOD_RES 12: note = PHOSPHORYLATION
RGGSQASSRSSSRS

SEQ ID NO: 8 - protein, 14 aa, synthetic construct
RGGSQASSRSSSRS

SEQ ID NO: 9 - protein, 6 aa, synthetic construct
MSDNGP

SEQ ID NO: 10 - protein, 9 aa, synthetic construct
APRITFGGP

SEQ ID NO: 11 - protein, 19 aa, synthetic construct
GPTDSTGSNQNGERSGARS

SEQ ID NO: 12 - protein, 9 aa, synthetic construct
ASWFTALTQ

SEQ ID NO: 13 - protein, 12 aa, synthetic construct
VPINTNSSPDDQ

SEQ ID NO: 14 - protein, 9 aa, synthetic construct
YRRATRRIR

SEQ ID NO: 15 - protein, 19 aa, synthetic construct
MKDLSPRWYFYYLGTGPEA

SEQ ID NO: 16 - protein, 9 aa, synthetic construct
GALNTPKDH

SEQ ID NO: 17 - protein, 9 aa, synthetic construct
PQGTTLPKG

SEQ ID NO: 18 - protein, 39 aa, synthetic construct
YAEGSRGGSQASSRSSSRSRNSSRNSTPGSSRGTSPARM

SEQ ID NO: 19 - protein, 48 aa, synthetic construct
ESKMSGKGQVTKKSAAEAGMEVTPAGAKKQQTVTLLQQSMSSADSTQA